(19) United States
(12) Patent Application Publication
(10) Pub. No.: US 2013/0178479 A1
(43) Pub. Date: Jul. 11, 2013

(54) PHARMACEUTICAL COMBINATIONS

(71) Applicants: Yan Chen, Lexington, MA (US); Xizhong Huang, Southborough, MA (US); Leon Murphy, Newton, MA (US); Beat Nyfeller, Cambridge, MA (US)

(72) Inventors: Yan Chen, Lexington, MA (US); Xizhong Huang, Southborough, MA (US); Leon Murphy, Newton, MA (US); Beat Nyfeller, Cambridge, MA (US)

(73) Assignee: NOVARTIS AG, Basel (CH)

(21) Appl. No.: 13/876,021

(22) PCT Filed: Oct. 3, 2011
(86) PCT No.: PCT/US11/54536

§ 371 Date: Mar. 26, 2013

Related U.S. Application Data
(60) Provisional application No. 61/389,445, filed on Oct. 4, 2010.

(51) Int. Cl. A61K 31/436; A61K 31/496; A61K 31/4745

(52) U.S. Cl. CPC A61K 31/436; A61K 31/4745; A61K 31/496
USPC 514/253.03; 514/293

(57) ABSTRACT

The present invention relates to a pharmaceutical combination which comprises (a) an mTOR catalytic inhibitor, such as a catalytic phosphatidylinositol-3-kinase (PI3K) and mTOR inhibitor compound which is an imidazoquinoline derivative and (b) at least one allosteric mTOR inhibitor compound, and optionally, at least one pharmaceutically acceptable carrier for simultaneous, separate or sequential use, in particular for the treatment of an mammalian target of rapamycin (mTOR) kinase dependent proliferative diseases; and the uses of such a combination in the treatment of mTOR kinase dependent proliferative diseases; a pharmaceutical composition comprising such a combination; the use of such a combination for the preparation of a medicament for the treatment of a proliferative disease; a commercial package or product comprising such a combination as a combined preparation for simultaneous, separate or sequential use; and to a method of treatment of a warm-blooded animal, especially a human.

### FIELD OF THE INVENTION

The present invention relates to a pharmaceutical combination comprising (a) a catalytic phosphatidylinositol-3-kinase (PI3K)/mammalian target of rapamycin (mTOR) inhibitor compound which is an imidazoquinoline derivative of formula (I) and (b) at least one allosteric mTOR inhibitor compound, and optionally, at least one pharmaceutically acceptable carrier for simultaneous, separate or sequential use; and the uses of such a combination in the treatment of a proliferative disease, more specifically a mTOR kinase dependent proliferative disease; a pharmaceutical composition comprising such a combination; the use of such a combination for the preparation of a medicament for the treatment of a proliferative disease, more specifically a mTOR kinase dependent proliferative disease; a method of treatment of a subject in need thereof, especially a human; and a commercial package or product comprising such a combination as a combined preparation for simultaneous, separate or sequential use.

### BACKGROUND OF THE INVENTION

In mammalian cells, the target of rapamycin (mTOR) kinase exists as a multiprotein complex described as the mTORC1 complex or mTORC2 complex, which senses the availability of nutrients and energy and integrates inputs from growth factors and stress signaling. The mTORC1 complex is sensitive to allosteric mTOR inhibitor compounds (such as rapamycin); is composed of mTOR, GβL, and regulatory associated proteins of mTOR (raptor); and binds to the peptidyl-prolyl isomerase FKBP12 protein (a FK506-binding protein 1A, 12 kDa). In contrast, the mTORC2 complex is composed of mTOR, GβL, and rapamycin-insensitive companion proteins of mTOR (rictor) and does not bind to the FKBP12 protein in vitro.

The mTORC1 complex has been shown to be involved in protein translational control, operating as a growth factor and nutrient sensitive apparatus for growth and proliferation regulation. mTORC1 regulates protein translation via two key downstream substrates; S6 kinase, which in turn phosphorylates ribosomal protein S6, and eukaryotic translation initiation factor 4E binding protein 1 (4EBP1), which plays a key role in modulating eIF4E regulated cap-dependent translation. The mTORC1 complex regulates cell growth in response to the energy and nutrient homeostasis of the cell, and the deregulation of the mTORC1 complex is common in a wide variety of human cancers. The function of mTORC2 involves the regulation of cell survival via phosphorylation of Akt (Sarbassov et al., Science, 2005, 307: 1098-1101) and the modulation of actin cytoskeleton dynamics (Jacinto et al., Nat. Cell. Biol., 2004, 6: 1122-1128).

The mTORC1 complex is sensitive to allosteric mTOR inhibitor compounds, such as rapamycin and derivatives thereof, in large part due to the allosteric mTOR inhibitor compound's mode of action, which involves the formation of an intracellular complex with the FKBP12 and binding to the FKBP12-rapamycin binding (FRB) domain of mTOR (Choi et al., Science, 1996, 273:239-242). This results in a conformational change in mTORC1 which is believed to alter and weaken the interaction with its scaffolding protein raptor, in turn impeding substrates such as S6K1 from accessing mTOR and being phosphorylated (Hara et al., Cell, 2002, 110(2): 177-89; Kim et al., Cell, 2002, 110(2): 183-75: Oshiro et al., Genes Cells, 2004, 9(4): 359-66). Rapamycin and rapalogues such as RAD001 or CCI-779 have gained clinical relevance by inhibiting hyperactivation of mTOR associated with both benign and malignant proliferation disorders (Dancey, Nature Reviews Clinical Oncology, 2010, 7; 209-219; Hidalgo and Rowinsky, Oncogene, 2000, 19:6680-6686).

Everolimus (Afinitor®, Novartis) is an FDA approved drug for the treatment of advanced kidney cancer and is still being investigated in several other phase III clinical trials in oncology. Preclinical studies have shown that Everolimus is able to inhibit the proliferation of a wide variety of tumor cell lines both in vitro and in vivo, presumably through the suppression of rapamycin sensitive mTORC1 function. Everolimus, as a derivative of rapamycin, is an allosteric mTOR inhibitor compound that is highly potent at inhibiting part of the mTORC1 function, namely S6 kinase (S6K) and the downstream S6K substrate S6. However, everolimus (and other rapamycin analogues) has little or no effect at inhibiting the priming phosphorylation phosphorylation events in 4EBP1 (T37/46), which has recently been implicated in Hsieh et al., Cancer Cell, 17(3): 249-261 (2010) as a key driver in tumorigeneses and maintenance. And allosteric mTOR inhibitor compounds like everolimus (and other rapamycin analogues) have little or no effect at inhibiting the mTORC2 pathway, or its resulting activation of Akt signaling.

In contrast, catalytic, ATP-competitive mTOR inhibitor compounds have been found to target the mTOR kinase domain directly and target both mTORC1 and mTORC2 (Feldman et al., PLoS Biology, 2009, 7(2): e1000038; Garcia-Martinez et al., Biochem. J., 2009, 421(Pt. 1): 29-42; Thoreen et al., J. Biol. Chem., 2009, 284: 8023-8032; Yu et al, Cancer Res., 2009; 69; 6232). These are more effective inhibitors of mTORC1 than such allosteric mTOR inhibitor compounds, such as rapamycin, because they modulate rapamycin-resistant mTORC1 outputs such as 4EBP1-T37/46 phosphorylation and cap-dependent translation.

Specific imidazoquinoline derivatives and their preparation have been described in WO2006/122806 and include compounds of formula (I)

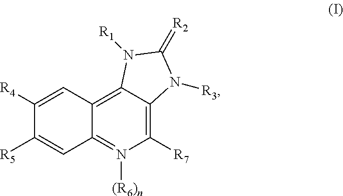

wherein R₁, R₂, R₃, R₄, n, R₆ and R₇ defined as set forth herein, or a tautomer thereof, or a pharmaceutically acceptable salt, or a hydrate or solvate thereof. Such imidazoquinoline derivatives, such as 8-(6-methoxy-pyridin-3-yl)-3-methyl-1-(4-piperazin-1-yl-3-trifluoromethyl-phenyl)-1,3-dihydro-imidazo[4,5-c]quinolin-2-one (“Compound A”) are proven to be effective PI3K/mTOR inhibitors, e.g., WO2008/103636 and Maira et al, Mol. Cancer Ther., 7(7): 1851-1863 (July 2008), which display broad activity against a large panel of cultured human cancer cell lines.

Acting as a catalytic PI3K/mTOR inhibitor, the imidazoquinoline derivative compound 2-methyl-2-[4-(3-methyl-2-oxo-8-quinolin-3-yl-2,3-dihydro-imidazo[4,5-c]quinolin-1-yl)-phenyl]-propionitrile is capable of shutting down the complete function of mTORC1 complex, including both the rapamycin sensitive (phosphorylation of S6K, and subsequently phosphorylation of S6) and rapamycin insensitive (phosphorylation of 4EBP1) functions. The imidazoquinoline derivative compound 2-methyl-2-[4-(3-methyl-2-oxo-8-quinolin-3-yl-2,3-dihydro-imidazo[4,5-c]quinolin-1-yl)-phenyl]-propionitrile has a differential effect according to the drug concentration used, whereby mTOR inhibition predominates at a low concentration (less than 100 nmol/L) but dual PI3K/mTOR inhibition is observed at relatively higher concentrations (approximately 500 nmol/L). (E.g. Serra et al, Cancer Res., 68(19): 8022-8030 (Oct. 1, 2008).)

In spite of numerous treatment options for patients with proliferative diseases, there remains a need for effective and safe therapeutic agents which can be administered to subjects in need thereof at low doses and a need for their preferential use in combination therapy. It has been surprisingly discovered that the combination of low amounts of the compound of formula (I) with low amounts of an allosteric mTOR inhibitor compound, such as everolimus, results in unexpected improvement in the treatment of tumor diseases. When administered simultaneously, sequentially or separately, the compound of formula (I) and the allosteric mTOR inhibitor compound interact in a synergistic manner to inhibit cell proliferation. This unexpected synergistic interaction allows a reduction in the dose required of each compound, leading to a reduction in the side effects and enhancement of the clinical effectiveness off the compounds and treatment.

### SUMMARY OF THE INVENTION

The present invention relates to a novel pharmaceutical combination comprising (a) a compound of formula (I)

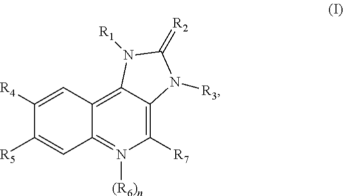

wherein
R₁ is naphthyl or phenyl wherein said phenyl is substituted by one or two substituents independently selected from the group consisting of Halogen; lower alkyl unsubstituted or substituted by halogen, cyano, imidazolyl or triazolyl; cycloalkyl; amino substituted by one or two substituents independently selected from the group consisting of lower alkyl, lower alkyl sulfonyl, lower alkoxy and lower alkoxy lower alkylamino; piperazinyl unsubstituted or substituted by one or two substituents independently selected from the group consisting of lower alkyl and lower alkyl sulfonyl; 2-oxo-pyrrolidinyl; lower alkoxy lower alkyl; imidazolyl; pyrazolyl; and triazolyl;

### R₂ is O or S;

R₃ is lower alkyl;
R₄ is pyridyl unsubstituted or substituted by halogen, cyano, lower alkyl lower alkoxy or piperazinyl unsubstituted or substituted by lower alkyl; pyrimidinyl unsubstituted or substituted by lower alkoxy; quinolinyl unsubstituted or substituted by halogen; quinoxalinyl; or phenyl substituted with alkoxy;
R₅ is hydrogen or halogen;
n is 0 or 1;
R₆ is oxide;
with the proviso that if n=1, the N-atom bearing the radical R₆ has a positive charge;
R₇ is hydrogen or amino;
or a tautomer thereof, or a pharmaceutically acceptable salt, or a hydrate or solvate thereof, and (b) at least one allosteric mTOR inhibitor compound and optionally at least one pharmaceutically acceptable carrier, wherein said compound of formula (I) is administered to a subject in need thereof in an amount between about 1 nM to about 100 nM or about 9.5×10⁻⁸ to about 9.5×10⁻⁶ Mole/kg or about 3 to about 315 mg/subject per daily dose, for simultaneous, separate or sequential use, in particular for the treatment of a proliferative disease, more specifically a mammalian target of rapamycin (mTOR) kinase dependent proliferative disease.

In a preferred embodiment, the COMBINATION OF THE INVENTION pertains to a pharmaceutical combination which comprises (a) the compound 2-methyl-2-[4-(3-methyl-2-oxo-8-quinolin-3-yl-2,3-dihydro-imidazo[4,5-c]quinolin-1-yl)-phenyl]-propionitrile (referred to as “Compound A” herein) or its monotosylate salt and (b) the allosteric mTOR inhibitor compound everolimus (RAD001), wherein Compound A is administered in an amount between about 1 nM to about 100 nM or about 9.5×10⁻⁸ to about 9.5×10⁻⁸ Mole/kg or about 3 to about 315 mg/subject per daily dose for the treatment of a mTOR kinase dependent proliferative disease. In a further embodiment, the allosteric mTOR inhibitor compound everolimus (RAD001) used in the combination is administered in a therapeutically effect amount from about 0.001 nM to about 17.8 nM or from about 8.5×10⁻¹² Mole/kg to about 1.5×10⁻⁷ Mole/kg, or from about 0.00056 mg/subject to about 10 mg/subject per daily dose.

In one aspect the present invention provides the use of a pharmaceutical combination which comprises (a) a compound of formula (I) or a tautomer thereof, or a pharmaceutically acceptable salt, or a hydrate or solvate thereof, and (b) at least one allosteric mTOR inhibitor compound and optionally at least one pharmaceutically acceptable carrier for the manufacture of a medicament for the treatment or prevention of a mTOR kinase dependent proliferative disease, wherein the compound of formula (I) is administered to a subject in need thereof in an amount between about 1 nM to about 100 nM or about 9.5×10⁻⁸ to about 9.5×10⁻⁶ Mole/kg or about 3 to about 315 mg/subject per daily dose.

In a preferred embodiment, the present invention pertains to a use of a pharmaceutical combination which comprises (a) Compound A or its monotosylate salt and (b) the allosteric mTOR inhibitor compound everolimus (RAD001), wherein Compound A is administered in an amount between about 1 nM to about 100 nM or about 9.5×10⁻⁸ to about 9.5×10⁻⁶ Mole/kg or about 3 to about 315 mg/subject per daily dose for the treatment of a mTOR kinase dependent proliferative disease. In a further embodiment, the allosteric mTOR inhibitor compound everolimus (RAD001) is administered in a therapeutically effect amount from about 0.001 nM to about 17.8 nM or from about 8.5×10⁻¹² Mole /kg to about 1.5×10⁻⁷ Mole/kg, or from about 0.00056 mg/subject to about 1.0 mg/subject per daily dose.

In another aspect, the present invention provides a method of treating or preventing a proliferative disease comprising administering (a) a therapeutically effective amount of a compound of formula (I) or a tautomer thereof, or a pharmaceutically acceptable salt, or a hydrate or solvate thereof, and (b) a therapeutically effective amount of at least one allosteric mTOR inhibitor compound and optionally at least one pharmaceutically acceptable carrier to a subject in need thereof, wherein the compound of formula (I) is administered in an amount between about 1 nM to about 100 nM or about 9.5×10⁻⁸ to about 9.5×10⁻⁶ Mole/kg or about 3 to about 315 mg/subject per daily dose. Preferably, the compound of formula (I) is Compound A.

In one aspect the invention provides a method for improving efficacy of the treatment of a mTOR kinase dependent proliferative disease, by administering (a) a compound of formula (I) or a tautomer thereof, or a pharmaceutically acceptable salt, or a hydrate or solvate thereof, and (b) at least one allosteric mTOR inhibitor compound and optionally at least one pharmaceutically acceptable carrier to a subject in need thereof, wherein the compound of formula (I) is administered to a subject in need thereof in an amount between about 1 nM to about 100 nM or about 9.5×10⁻⁸ to about 9.5×10⁻⁶ Mole/kg or about 3 to about 315 mg/subject per daily dose. Preferably, the compound of formula (I) is Compound A.

In one aspect of the invention, the present invention pertains to a pharmaceutical combination such as a combined preparation or a pharmaceutical composition comprising (a) a compound of formula (I), and (b) at least one allosteric mTOR inhibitor compound and optionally at least one pharmaceutically acceptable carrier for simultaneous, separate or sequential use, in particular for the treatment of an mTOR kinase dependent proliferative diseases, wherein the compound of formula (I) is administered to a subject in need thereof in an amount between about 1 nM to about 100 nM or about 9.5×10⁻⁸ to about 9.5×10⁻⁶ Mole/kg or about 3 to about 315 mg/subject per daily dose. Preferably, the compound of formula (I) is Compound A.

The present invention further provides a commercial package, comprising as active ingredients COMBINATION OF THE INVENTION, together with instructions for simultaneous, separate or sequential use thereof in the delay of progression or treatment of a proliferative disease.

### DETAILED DESCRIPTION OF THE FIGURES

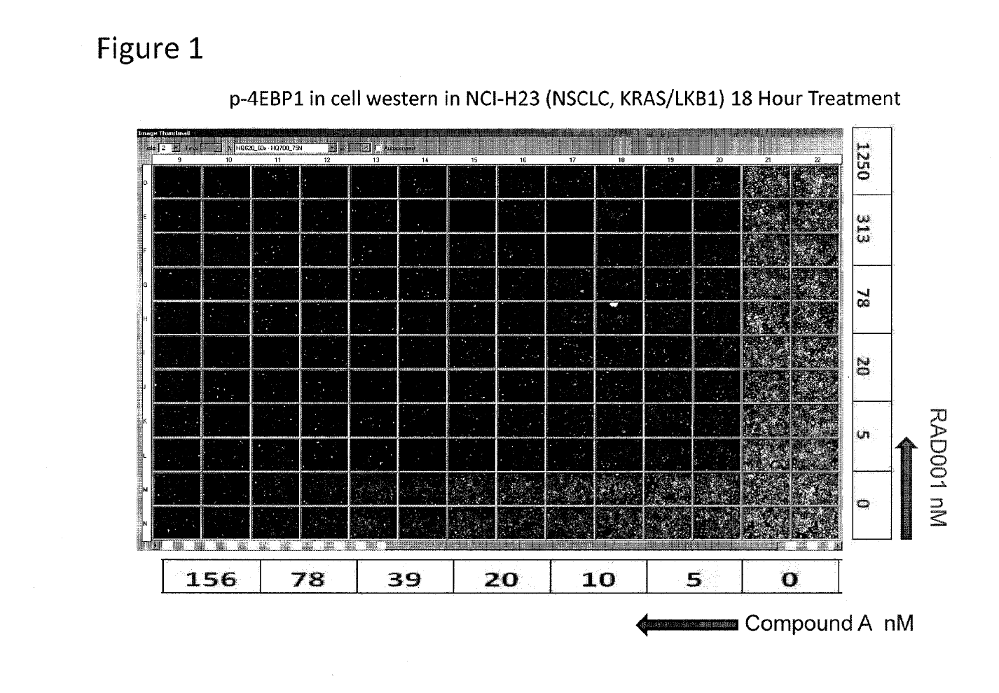

FIG. 1 shows effect of single agent and concomitant everolimus (RAD001 or PKF-222-6666-NX-2) and/or the catalytic PI3K/mTOR inhibitor compound 2-methyl-2-[4-(3-methyl-2-oxo-8-quinolin-3-yl-2,3-dihydro-imidazo[4,5-c]quinolin-1-yl)-phenyl]-propionitrile (Compound A) treatment on the phosphorylation of 4EBP1 in NCI-H23 (KRAS and LKB1 mutant) human non-small cell lung cancer cell models by immunofluorescence-based staining with a T37/46 phospho-specific antibody and automated imaging and quantitation (high content p4EBP1 T37/46 assay readout).

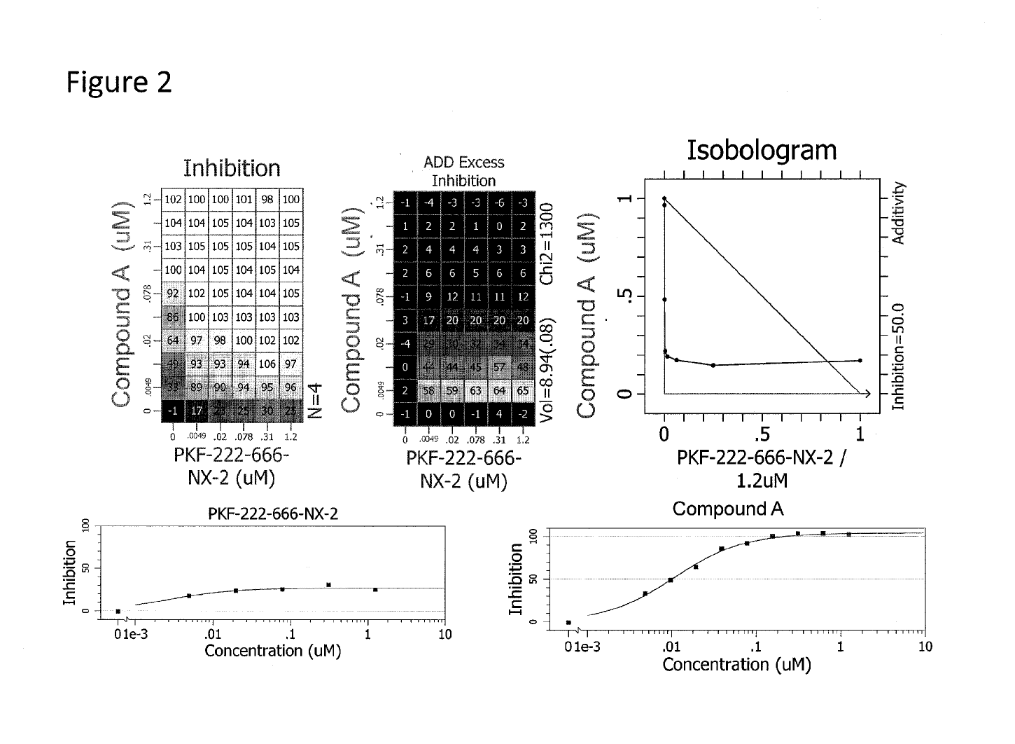

FIG. 2 shows full dose matrix data from the high content analysis of p-4EBP1 in NCI-H23 human non-small cell lung cancer cell models.

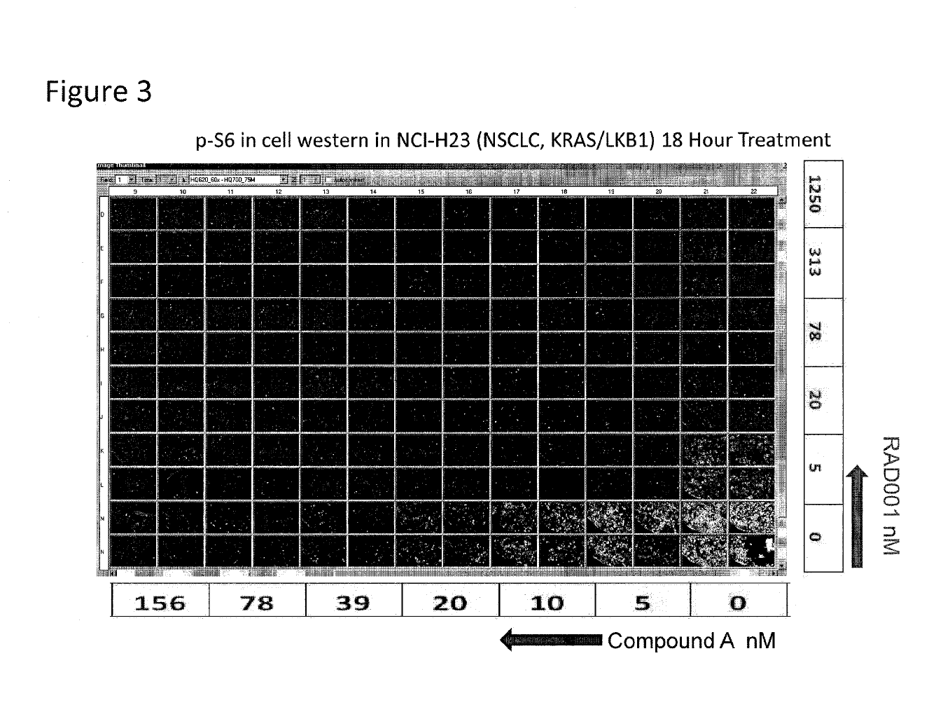

FIG. 3 shows effect of single agent and concomitant everolimus (RAD001) and/or Compound A treatment on the phosphorylation of S3 in NCI-H23 (KRAS and LKB1 mutant) human non-small cell lung cancer cell models using a high content pS6 S240/244 assay readout.

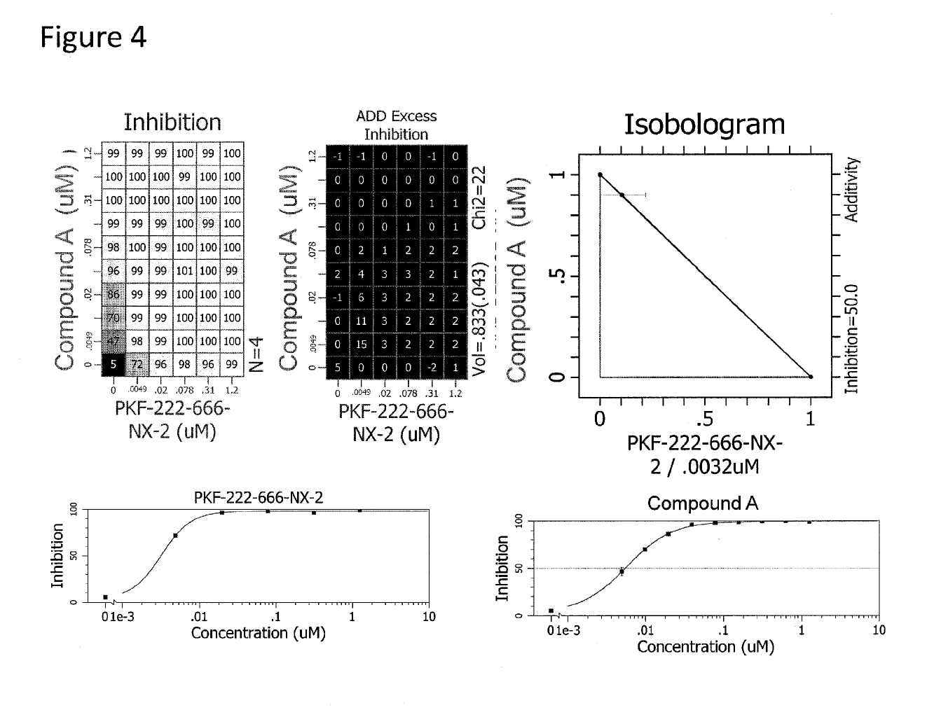

FIG. 4 shows full dose matrix data from the high content analysis of pS6 in NCI-H23 human non-small cell lung cancer cell models.

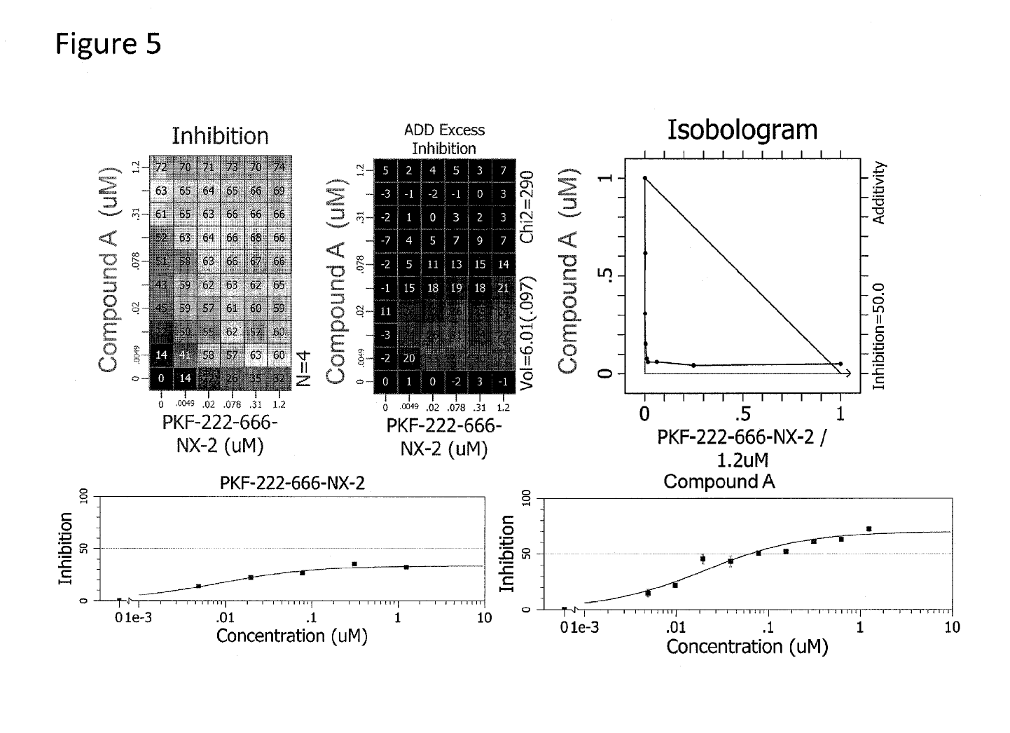

FIG. 5 shows full dose matrix cell proliferation data from single agent and concomitant everolimus (RAD001) and/or Compound A treatment in NCI-H23 human non-small cell lung cancer cell models.

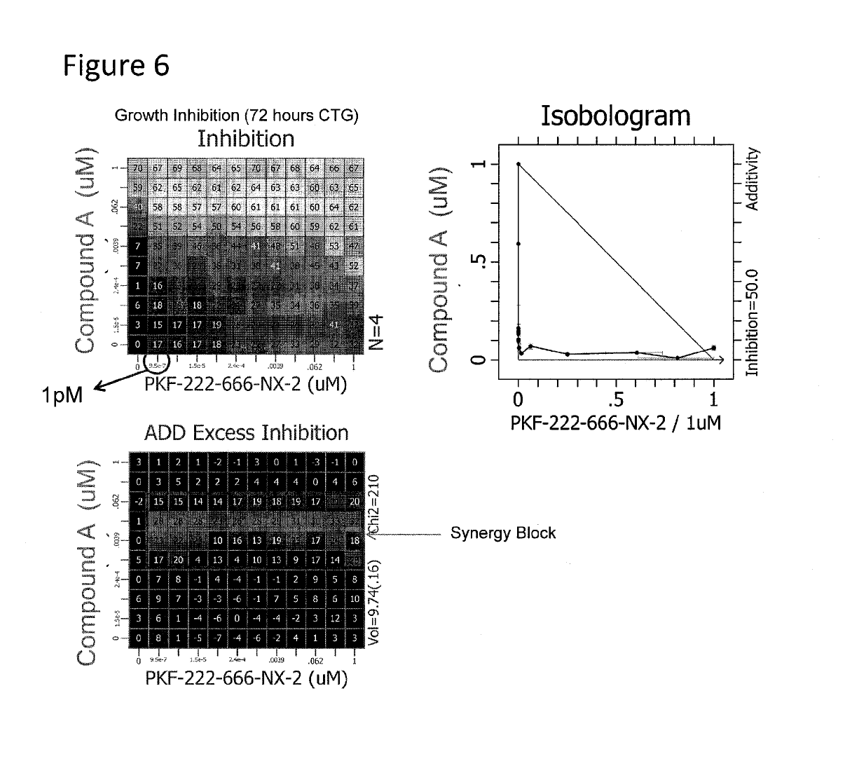

FIG. 6 shows effects of combining lower everolimus (RAD001) and Compound A doses on proliferation of NCI-H23 human non-small cell lung cancer cell models. In this extended dose matrix, as little as 1 pM everolimus is needed to shift the Compound A IC₅₀

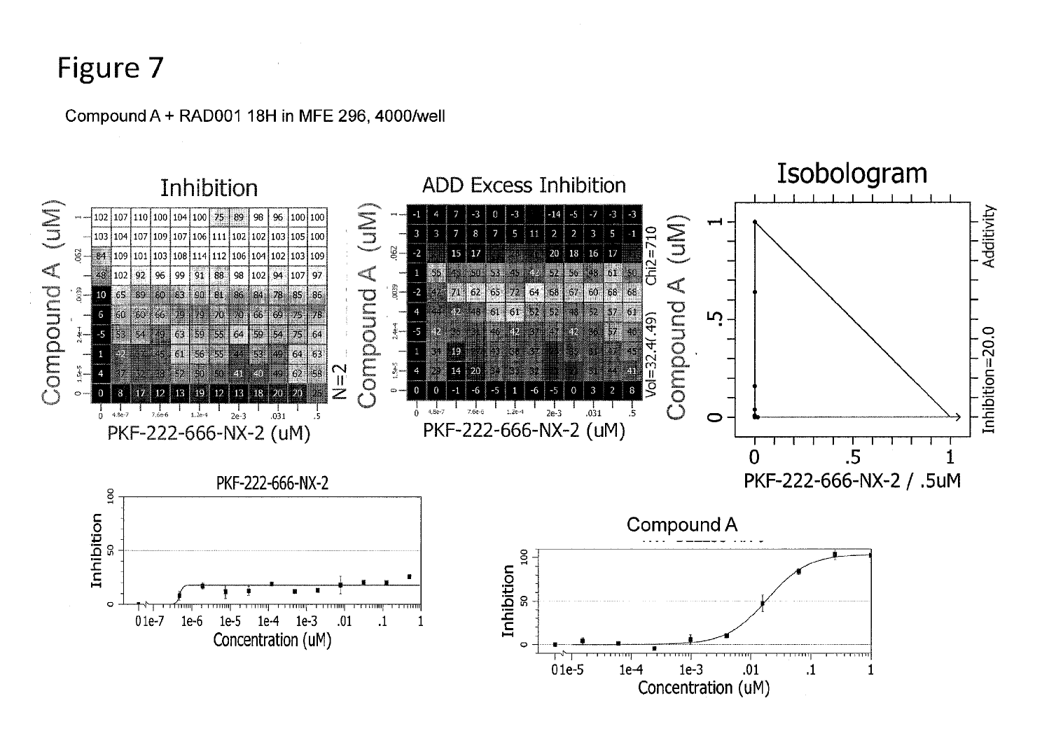

FIG. 7 shows full dose matrix data for single agent and concomitant everolimus (RAD001) and/or Compound A treatment on the phosphorylation of 4EBP1 in MFE296 (PIK3CA and PTEN mutant) human endometrial cancer cell models using a high content readout.

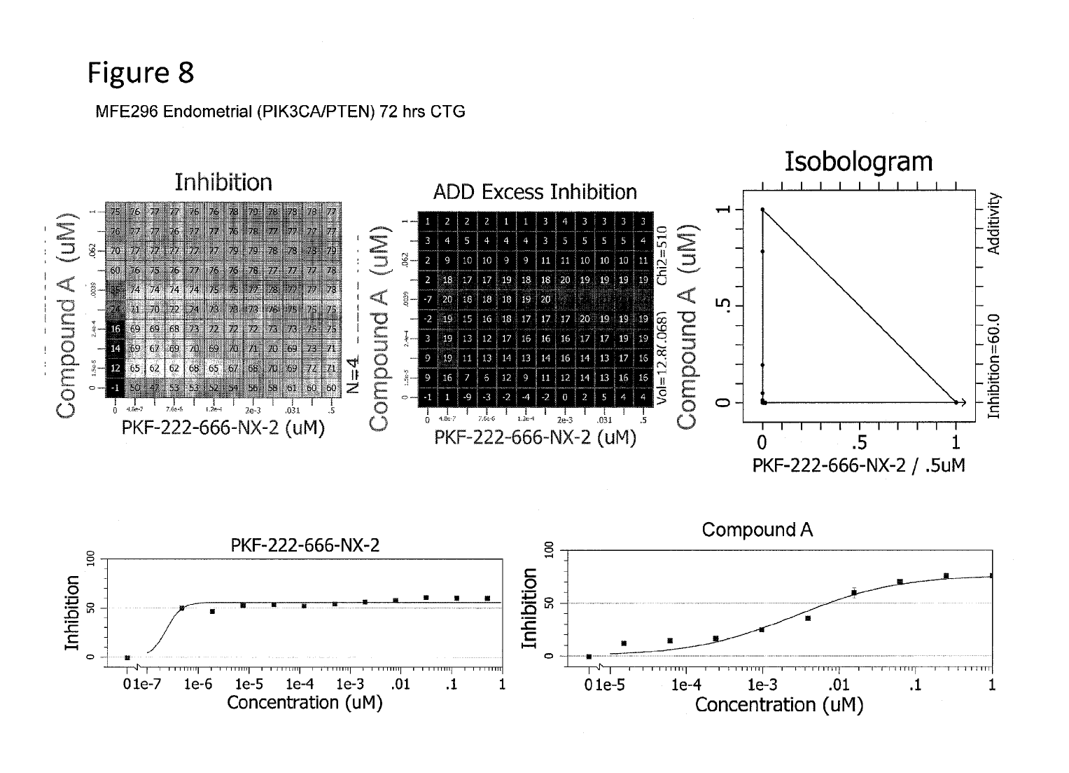

FIG. 8 shows extended dose matrix cell proliferation data for MFE296 (PIK3CA and PTEN mutant) human endometrial cancer cell models.

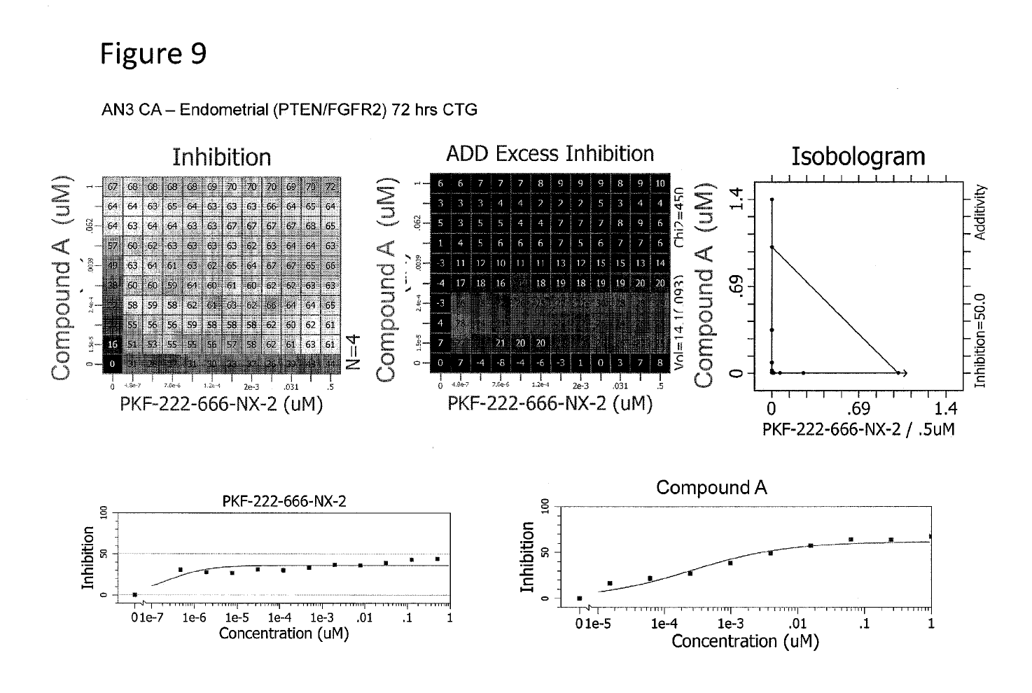

FIG. 9 shows extended dose matrix cell proliferation data for AN3 CA (FGFR2 and PTEN mutant) human endometrial cancer cell models.

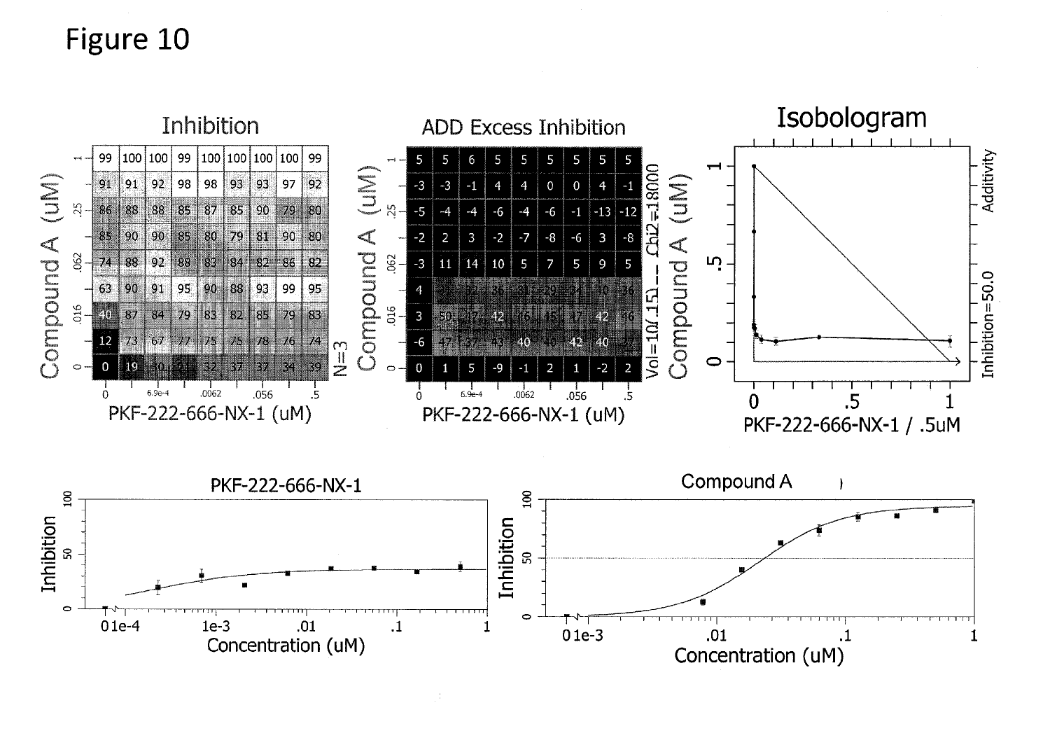

FIG. 10 shows extended dose matrix cell proliferation data for GA-10 human Non-Hodgkins Lymphoma cancer cell models.

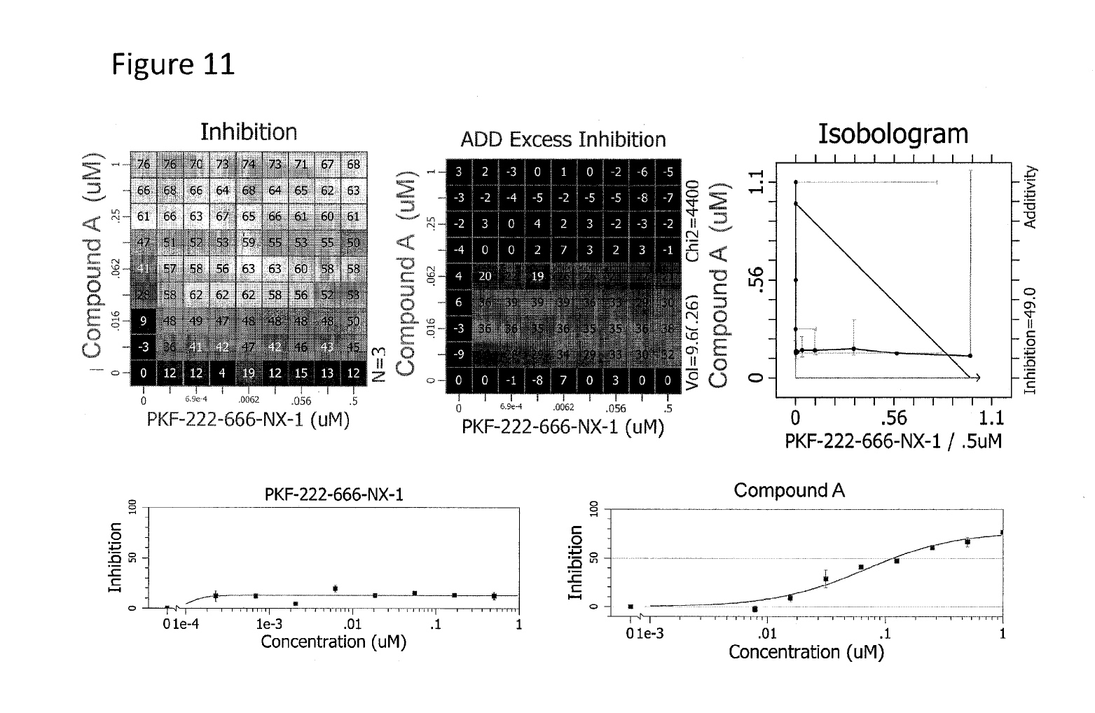

FIG. 11 shows extended dose matrix cell proliferation data for RPMI 8226 human Multiple Myeloma cancer cell models.

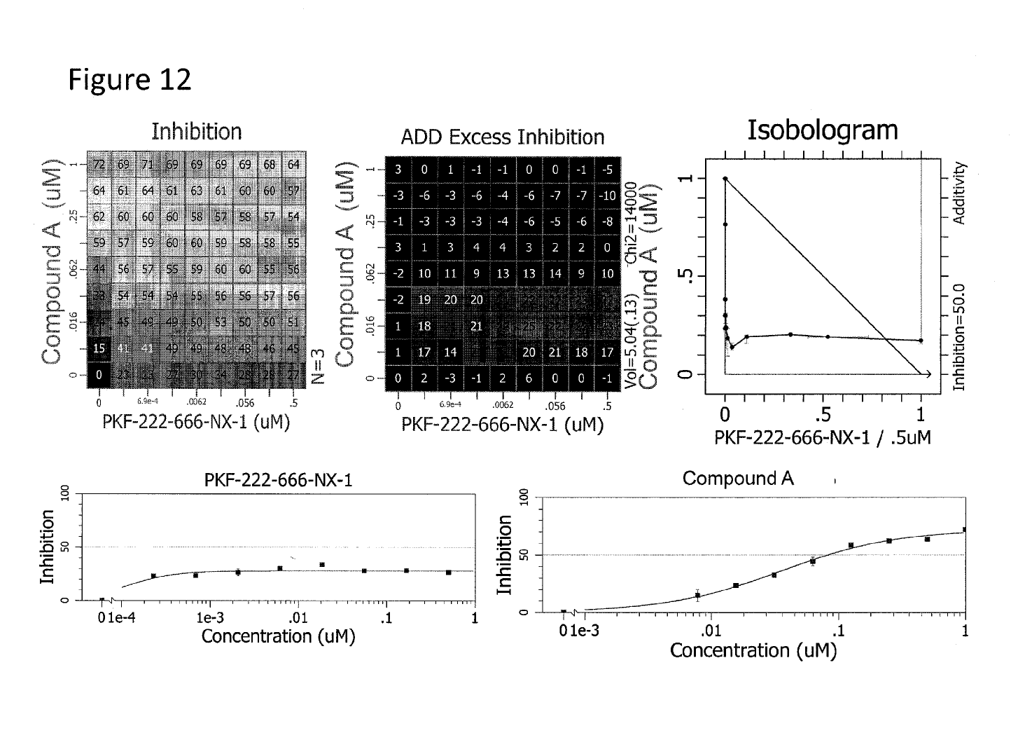

FIG. 12 shows extended dose matrix cell proliferation data for KMS-11 (FGFR3 mutant) human Multiple Myeloma cancer cell models.

### DETAILED DESCRIPTION OF THE INVENTION

Throughput this specification and in the claims that follow, the following terms are defined with the following meanings, unless explicitly stated otherwise;

The terms “comprising” and “including” are used herein in their open, non-limiting sense.

Where the plural form is used for compounds, salts, and the like, this is taken to mean also a single compound, salt, or the like.

The term “combination” is defined to refer to either a fixed combination in one dosage unit form, or a non-fixed combination (or a kit of parts) for the combined administration where compound of formula (I), and a combination partner may be administered independently at the same time or separately within time intervals that allow that the combination partners show a cooperative, e.g., synergistic, effect. The term “combined administration” or the like as utilized herein are meant to encompass administration of the selected combination partner to a single subject in need thereof (e.g. a patient), and are intended to include treatment regimens in which the agents are not necessarily administered by the same route of administration or at the same time, The term “fixed combination” means that the active ingredients, e.g. a compound of formula (I) and a combination partner, are both administered to a patient simultaneously in the form of a single entity or dosage. The term “non-fixed combination” mean that the active ingredients, e.g. a compound of formula (I) and a combination partner, are both administered to a patient as separate entities either simultaneously, concurrently or sequentially with no specific time limits, wherein such administration provides therapeutically effective levels of the two compounds in the body of the-patient. The latter also applies to cocktail therapy, e.g. the administration of three or more active ingredients.

The term “catalytic PI3K/mTOR inhibitors” is defined herein as compounds which target, decrease or inhibit the catalytic activity/function of the PBK and/or mTOR enzymes by binding to the ATP binding cleft of these enzymes.

The term “allosteric mTOR inhibitor compounds” is defined herein as compounds which target, decrease or inhibit the activity/function of the mTOR kinase through binding to an allosteric binding site, preferably the FKBP12-rapamycin binding site (FRB), of the mTORC1 complex.

The term “subject” is intended to include animals. Examples of subjects include mammals, e.g., humans, dogs, cows, horses, pigs, sheep, goats, cats, mice, rabbits, rats, and transgenic non-human animals, in certain embodiments, the subject is a human, e.g., a human suffering from, at risk of suffering from, or potentially capable of suffering from a brain tumor disease.

The term “mg/subject” is defined herein to be the amount of the referenced compound in milligrams as estimated for a subject in need thereof having approximately 70 kg body mass. If is understood that this term is not restricted to a subject haying approximately 70 kg body mass and the amount of the referenced in milligrams would be adjusted by one of ordinary skill to be equivalent to this ratio at the actual body mass of the subject.

The term “about” in connection with a particular drug dose shall have the meaning of a drug dose in the range of plus/minus 10% w/w, preferably plus/minus 5% w/w or less, of the nominal drug dose. By way of example, a nominal drug dose of about 0.01 mg active ingredient may contain from 0.009 to 0.011 mg, preferably from 0.0095 to 0.0105 active ingredient per dose.

The term “pharmaceutical composition” is defined herein to refer to a mixture or solution containing at least one therapeutic compound to be administered to a mammal, e.g., a human in order to prevent, treat or control a particular disease or condition affecting the mammal.

The term “pharmaceutically acceptable” is defined herein to refer to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

The term “a combined preparation” as used herein defines especially a “kit of parts” in the sense that the combination partners (a) and (b) as defined above can be dosed independently or by use of different fixed combinations with distinguished amounts of the combination partners (a) and (b), i.e. simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g. in order to cope with the needs of a patient sub-population to be treated or the needs of the single.

The term “pharmaceutical composition” as used herein shall refer to tor example, a mixture containing a specified amount of a therapeutic compound, e.g. an amount, of a therapeutic compound in a pharmaceutically acceptable carrier to be administered to a mammal, e.g., a human in order to treat mTOR kinase dependent proliferative diseases.

The term “treating” or “treatment” as used herein comprises a treatment effecting a delay of progression of a disease. The term “delay of progression” as used herein means administration of the combination to patients being in a pre-stage or in an early phase of the proliferative disease to be treated, in which patients for example a pre-form of the corresponding disease is diagnosed or which patients are in a condition, e.g. during medical treatment or a condition resulting from an accident, under which it is likely that a corresponding disease will develop.

The term “mTOR kinase dependent proliferative diseases” as used herein is defined to refer to any proliferative disease or disorder mentioned herein is meant; particularly any proliferative disease is meant that responds to the referenced compounds which inhibits the mTOR kinase pathway, especially, a proliferative disease selected from a cancer or tumor disease.

“Therapeutically effective” or “clinically effective” preferably relates to an amount that is therapeutically or in a broader sense also prophylactically effective against the progression of a proliferative disease.

“Jointly therapeutically active” or “joint therapeutic effect” means that the compounds may be given separately (in a chronically staggered manner, especially a sequence-specific manner) in such time intervals that they preferably, in the warm-blooded animal, especially human, to be treated, still show a (preferably synergistic) interaction (joint therapeutic effect). Whether this is the case, can inter alia be determined by following the blood levels, showing that both compounds are present in the blood of the human to be treated at least during certain time intervals.

The present invention relates to a novel pharmaceutical combination comprising (a) a compound of formula (I) or a tautomer thereof, or a pharmaceutically acceptable salt, or a hydrate or solvate thereof, and (b) at least one allosteric mTOR inhibitor compound and optionally at least one pharmaceutically acceptable carrier for simultaneous, separate or sequential use, in particular for the treatment of a proliferative disease, more specifically a mTOR kinase dependent proliferative disease, wherein the compound of formula (I) is administered to a subject in need thereof in an amount between about 1 nM to about 100 nM or about 9.5×10⁻⁸ to about 9.5×10⁻⁵ Mole/kg or about 3 to about 315 mg/subject per daily dose.

A combination which comprises (a) a compound of formula (I) or a tautomer thereof, or a pharmaceutically acceptable salt, or a hydrate or solvate thereof, and (b) at least one allosteric mTOR inhibitor compound and optionally at least one pharmaceutically acceptable carrier, wherein the compound of formula (I) is administered to a subject in need thereof in an amount between about 1 nM to about 100 nM or about 9.5×10⁻⁸ to about 9.5×10⁻⁶ Mole/kg or about 3 to about 315 mg/subject per daily dose, will be referred to hereinafter as a COMBINATION OF THE INVENTION.

Surprisingly, it has been discovered that the combination of low amounts (from about 1 nM to about 100 nM or from about 9.5×10⁻⁸ to about 9.5×10⁻⁶ Mole/kg or from about 3 to about 315 mg/person) of the compound of formula (I), with low amounts (from 0.001 nM to about 17.8 nM or from about 8.5×10⁻¹² Mole/kg to about 1.5×10⁻⁷ Mole/kg, or from about 0.00056 mg/subject to about 10 mg/subject) of at least one allosteric mTOR. inhibitor compound, such as everolimus, results in unexpected improvement in the treatment of proliferative diseases, particularly mTOR kinase dependent proliferative diseases. When administered simultaneously, sequentially or separately, the compound of formula (I) and the allosteric mTOR inhibitor compound interact in a synergistic manner to inhibit the phosphorylation of 4EBP1 and cell proliferation. This unexpected synergistic interaction allows a reduction in the dose required of each compound, leading to a reduction in the side effects and enhancement of the clinical effectiveness of the compounds and treatment. The foregoing COMBINATION OF THE INVENTION is capable of enhancing the inhibition of proliferation of cancer cells to the range where, as a single agent, only high doses (from about 250 nM to about 1000 nM, or from about 2.4×10⁻⁵ to 9.5×10⁻⁵ Mole/kg, or about 784 to 3136 mg/subject) of the compound of formula (I) can only achieve.

Determining a synergistic interaction between one or more components, the optimum range for the effect and absolute dose amounts of each component for the effect may be definitively measured by administration of the components over different w/w ratio ranges and doses to patients in need of treatment. For humans, the complexity and cost of carrying out clinical studies on patients renders impractical the use of this form of testing as a primary model for synergy. However, the observation of synergy in one species can be predictive of the effect in other species and animal models exist, as described herein, to measure a synergistic effect and the results of such studies can also be used to predict effective dose and plasma concentration ratio ranges and the absolute doses and plasma concentrations required in other species by the application of pharmacokinetic/pharmacodynamic methods. Established correlations between tumor models and effects seen in man suggest that synergy in animals may, e.g., be demonstrated in the NCI-H23 human non-small cell lung cancer tumor model, the MFE 296 human endometrial cancer cell model (which carries both PIK3CA and PTEN mutations) and AN3CA endometrial cancer cell model, the KMS11 and RPMI 8226 myeloma cancer cell mode), and GA-10 non-Hodgkin's B cell lymphoma cancer cell model as described in the Examples below.

The COMBINATION OF THE INVENTION includes a catalytic PI3K/mTOR inhibitor. Catalytic PI3K/mTOR inhibitor compounds suitable for the present invention include compounds of formula (I)

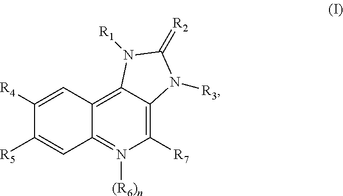

wherein
R₁ is naphthyl or phenyl wherein said phenyl is substituted by one or two substituents independently selected from the group consisting of Halogen; lower alkyl unsubstituted or substituted by halogen, cyano, imidazolyl or triazolyl; cycloalkyl; amino substituted by one or two substituents independently selected from the group consisting of lower alkyl, lower alkyl sulfonyl, lower alkoxy and lower alkoxy lower alkylamino; piperazinyl unsubstituted or substituted by one or two substituents independently selected from the group consisting of lower alkyl and lower alkyl sulfonyl; 2-oxo-pyrrolidinyl; lower alkoxy lower alkyl; imidazolyl; pyrazolyl; and triazolyl;

### R₂ is O or S;

R₃ is lower alkyl;
R₄ is pyridyl unsubstituted or substituted by halogen, cyano, lower alkyl, lower alkoxy or piperazinyl unsubstituted or substituted by lower alkyl; pyrimidinyl unsubstituted or substituted by lower alkoxy; quinolinyl unsubstituted or substituted by halogen; quinoxalinyl; or phenyl substituted with alkoxy
R₅ is hydrogen or halogen;
n is 0 or 1;
R₆ is oxido;
with the proviso that if n=1, the N-atom bearing the radical R₆ has a positive charge;
R₇ is hydrogen or amino;
or a tautomer thereof, or a pharmaceutically acceptable salt, or a hydrate or solvate thereof. These specific imidazoquinoline derivatives suitable for the present invention, their preparation and suitable pharmaceutical formulations containing the same are described in WO2006/122806, which is hereby incorporated by reference hereto in its entirety.

The radicals and symbols as used in the definition of a compound of formula (I) have the meanings as disclosed in WO2006/122806. The following general definitions shall apply in this specification, unless otherwise specified:

“Lower” shall refer to a radical having up to and including a maximum of 7, especially up to and including a maximum of 4 carbon atoms, the radicals in question being either linear or branched with single or multiple branching.

In a preferred embodiment, alkyl has up to a maximum of 12 carbon atoms and is especially lower alkyl.

“Lower alkyl” is preferably alkyl with from and including 1 up to and including 7, preferably from and including 1 to and including 4, and is linear or branched: preferably; lower alkyl is butyl, such as n-butyl, sec-butyl isobutyl, tert-butyl, propyl, such as n-propyl or isopropyl, ethyl or preferably methyl.

“Cycloalkyl” is preferably cycloalkyl with from and including 3 up to and including 6 carbon atoms in the ring; cycloalkyl is preferably cyclopropyl, cyclobutyl, cyclopently or cyclohexyl.

“Alkyl” which is substituted by halogen is preferably perfluoro alkyl such as trifluoromethyl.

“Halogen” is especially fluorine, chlorine, bromine, or iodine, especially fluorine, chlorine, or bromine.

Salts of the compounds of formula (I) may be used in accordance with the present invention. Such salts are formed, for example, as acid addition salts, preferably with organic or inorganic acids, from compounds of formula (I) with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic add, decanoic acid, dodecanoic acid, glyeolic acid, lactic acid, tumeric acid, succinic acid, malonic acid, adipic acid, pimelic acid, suberic acid, azelaic acid, malic acid, tartaric acid, citric acid, amino acids, such as glutamic add or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonlc acid, 4-toluenesulfonic acid, 2-naphthalenesulfonic acid, 1,5-naphthalene-disulfonic acid, 2- or 3-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

A preferred compound of the present invention is a compound—described in WO2006/122806—chosen from the group consisting of;

2-Methyl-2-[4-(3-methyl-2-oxo-8-pyridin-4-yl-2,3-dihydro-imidazo[4,5-c]quinolin-1-yl)-phenyl]-propionitrile;
2-Methyl-2-[4-(3-methyl-2-oxo-8-pyridin-3-yl-2,3-dihydro-imidazo[4,5-c]quinolin-1-yl)-phenyl]-propionitrile;
2-{4-[8-(6-Methoxy-pyridin-3-yl)-3-methyl-2-oxo-2,3-dihydro-imidazo[4,5-c]quinolin-1-yl]-phenyl}-2-methyl-propionitrile;
2-{4-[8-(5-Methoxy-pyridin-3-yl)-3-methyl-2-oxo-2,3-dihydro-imidazo[4,5-c]quinolin-1-yl]-phenyl}-2-methyl-propionitrile;
2-Methyl-2-{4-[3-methyl-2-oxo-8-(6-piperazin-1-yl-pyridin-3-yl)-2,3-dihydro-imidazo[4,5-c]quinolin-1-yl]-phenyl}-propionitrile;
2-Methyl-2-(4-{3-methyl-8-[2-(4-methyl-piperazin-1-yl)-pyridin-4-yl]-2-oxo-2,3-dihydro-imidazo[4,5-c]quinolin-1-yl}-phenyl)-propionitrile;
2-Methyl-2-[4-(3-methyl-2-oxo-8-quinolin-3-yl-2,3-dihydro-imidazo[4,5-c]quinolin-1-yl)-phenyl]-propionitrile;
2-{4-[8-(2-Fluoro-quinolin-3-yl)-3-methyl-2-oxo-2,3-dihydro-imidazo[4,5-c]quinolin-1-yl]-phenyl}-2-methyl-propionitrile;
2-Methyl-2-[4-(3-methyl-2-oxo-8-quinolin-6-yl-2,3-dihydro-imidazo[4,5-c]quinolin-1-yl)-phenyl]-propionitrile;
2-Methyl-2-[4-(3-methyl-2-oxo-8-quinolin-5-yl-2,3-dihydro-imidazo[4,5-c]quinolin-1-yl)-phenyl]-propionitrile;
2-Methyl-2-[4-(3-methyl-2-oxo-8-quinoxalin-6-yl-2,3-dihydro-imidazo[4,5-c]quinolin-1-yl)-phenyl]-propionitrile;
2-Ethyl-2-[4-(3-methyl-2-oxo-8-pyridin-3-yl-2,3-dihydro-imidazo[4,5-c]quinolin-1-yl)-phenyl]-butyronitrile;
2-Ethyl-2-[4-(3-methyl-2-oxo-8-quinolin-3-yl-2,3-dihydro-imidazo[4,5-c]quinolin-1-yl)-phenyl]-butyronitrile;
1-[3-Fluoro-4-(2-oxo-pyrrolidin-1-yl)-phenyl]-3-methyl-8-pyridin-3-yl-1,3-dihydro-imidazo[4,5-c]quinolin-2-one;
1-[3-Fluoro-4-(2-oxo-pyrrolidin-1-yl)-phenyl]-3-methyl-8-quinolin-3-yl-1,3-dihydro-imidazo[4,5-c]quinolin-2-one;
3-Methyl-1-[4-(2-oxo-pyrrolidin-1-yl)-phenyl]-8-pyridin-3-yl-1,3-dihydro-imidazo[4,5-c]quinolin-2-one;
3-Methyl-1-[4-(2-oxo-pyrrolidin-1-yl)-phenyl]-8-quinolin-3-yl-1,3-dihydro-imidazo[4,5-c]quinolin-2-one;
1-{4-[Bis-(2-methoxy-ethyl)-amino]-3-fluoro-phenyl}-3-methyl-8-pyridin-3-yl-1,3-dihydro-imidazo[4,5-c]quinolin-2-one;
1-{4-[Bis-(2-methoxy-ethyl)-amino]-3-fluoro-phenyl}-3-methyl-8-quinolin-3-yl-1,3-dihydro-imidazo[4,5-c]quinolin-2-one;
1-{4-[Bis-(2-methoxy-ethyl)-amino]-phenyl}-3-methyl-8-pyridin-3-yl-1,3-dihydro-imidazo[4,5-c]quinolin-2-one;
1-{4-[Bis-(2-methoxy-ethyl)-amino]-phenyl}-3-methyl-8-quinolin-3-yl-1,3-dihydro-imidazo[4,5-c]quinolin-2-one;
3-Methyl-1-naphthalen-2-yl-8-pyridin-3-yl-1,3-dihydro-imidazo[4,5-c]quinolin-2-one;
3-Methyl-1-naphthalen-2-yl-8-quinolin-3-yl-1,3-dihydro-imidazo[4,5-c]quinolin-2-one;
1-(2-Chloro-phenyl)-3-methyl-8-pyridin-3-yl-1,3-dihydro-imidazo[4,5-c]quinolin-2-one;
1-(2-Chloro-phenyl)-3-methyl-8-quinolin-3-yl-1,3-dihydro-imidazo[4,5-c]quinolin-2-one;
3-Methyl-8-pyridin-3-yl-1-o-tolyl-1,3-dihydro-imidazo[4,5-c]quinolin-2-one;
3-Methyl-8-quinolin-3-yl-1-o-tolyl-1,3-dihydro-imidazo[4,5-c]quinolin-2-one;
1-(2-Ethyl-phenyl)-3-methyl-8-pyridin-3-yl-1,3-dihydro-imidazo[4,5-c]quinolin-2-one;
1-(2-Ethyl-phenyl)-3-methyl-8-quinolin-3-yl-1,3-dihydro-imidazo[4,5-c]quinolin-2-one;
3-Methyl-8-pyridin-3-yl-1-(2-trifluoromethyl-phenyl)-1,3-dihydro-imidazo[4,5-c]quinolin-2-one;
3-Methyl-8-quinolin-3-yl-1-(2-trifluoromethyl-phenyl)-1,3-dihydro-imidazo[4,5-c]quinolin-2-one;
1-(4-Fluoro-2-methyl-phenyl)-3-methyl-8-quinolin-3-yl-1,3-dihydro-imidazo[4,5-c]quinolin-2-one;
1-(4-Fluoro-2-methyl-phenyl)-3-methyl-8-quinolin-3-yl-1,3-dihydro-imidazo[4,5-c]quinolin-2-one;
1-(2-Chloro-4-fluoro-phenyl)-3-methyl-8-pyridin-3-yl-1,3-dihydro-imidazo[4,5-c]quinolin-2-one;
1-(2-Chloro-4-fluoro-phenyl)-3-methyl-8-quinolin-3-yl-1,3-dihydro-imidazo[4,5-c]quinolin-2-one;
1-(3-Chloro-phenyl)-3-methyl-8-pyridin-3-yl-1,3-dihydro-imidazo[4,5-c]quinolin-2-one;
1-(3-Chloro-phenyl)-3-methyl-8-quinolin-3-yl-1,3-dihydro-imidazo[4,5-c]quinolin-2-one;
3-Methyl-8-pyridin-3-yl-1-(3-trifluoromethyl-phenyl)-1,3-dihydro-imidazo[4,5-c]quinolin-2-one;
3-Methyl-8-quinolin-3-yl-1-(3-trifluoromethyl-phenyl)-1,3-dihydro-imidazo[4,5-c]quinolin-2-one;
1-(4-Methoxymethyl-phenyl)-3-methyl-8-pyridin-3-yl-1,3-dihydro-imidazo[4,5-c]quinolin-2-one;
1-(4-Methoxymethyl-phenyl)-3-methyl-8-quinolin-3-yl-1,3-dihydro-imidazo[4,5-c]quinolin-2-one;
1-[2-Chloro-4-(2-methoxy-ethyl)-phenyl]-3-methyl-8-pyridin-3-yl-1,3-dihydro-imidazo[4,5-c]quinolin-2-one;
1-[2-Chloro-4-(2-methoxy-ethyl)-phenyl]-3-methyl-8-quinolin-3-yl-1,3-dihydro-imidazo[4,5-c]quinolin-2-one;
1-[4-(2-Methoxy-ethyl)-phenyl]-3-methyl-8-quinolin-3-yl-1,3-dihydro-imidazo[4,5-c]quinolin-2-one;
1-[4-(2-Methoxy-ethyl)-phenyl]-3-methyl-8-pyridin-3-yl-1,3-dihydro-imidazo[4,5-c]quinolin-2-one;
2-Methyl-2-[4-(3-methyl-2-oxo-5-oxy-8-pyridin-3-yl-2,3-dihydro-imidazo[4,5-c]quinolin-1-yl)-phenyl]-propionitrile;
2-Methyl-2-[4-(3-methyl-2-oxo-5-oxy-8-quinolin-3-yl-2,3-dihydro-imidazo[4,5-c]quinolin-1-yl)-phenyl]-propionitrile;
2-[4-(7-Fluoro-3-methyl-2-oxo-8-pyridin-3-yl-2,3-dihydro-imidazo[4,5-c]quinolin-1-yl)-phenyl]-propionitrile;
2-[4-(7-Fluoro-3-methyl-2-oxo-8-quinolin-3-yl-2,3-dihydro-imidazo[4,5-c]quinolin-1-yl)-phenyl]-propionitrile;
N-Methyl-N-[4-(3-methyl-2-oxo-8-pyridin-3-yl-2,3-dihydro-imidazo[4,5-c]quinolin-1-yl)-phenyl]-methanesulfonamide;
Methyl-[4-(3-methyl-2-oxo-8-pyridin-3-yl-2,3-dihydro-imidazo[4,5-c]quinolin-1-yl)-phenyl]-carbamic acid tert-butyl ester;
Ethanesulfonic acid methyl-[4-(3-methyl-2-oxo-8-pyridin-3-yl-2,3-dihydro-imidazo[4,5-c]quinolin-1-yl)-phenyl]-amide;
Ethanesulfonic acid methyl-[4-(3-methyl-2-oxo-8-quinolin-3-yl-2,3-dihydro-imidazo[4,5-c]quinolin-1-yl)-phenyl]-amide;
N-Ethyl-N-[4-(3-methyl-2-oxo-8-pyridin-3-yl-2,3-dihydro-imidazo[4,5-c]quinolin-1-yl)-phenyl]-methanesulfonamide;
N-Ethyl-N-[4-(3-methyl-2-oxo-8-quinolin-3-yl-2,3-dihydro-imidazo[4,5-c]quinolin-1-yl)-phenyl]-methanesulfonamide;
2-[4-(3-Ethyl-2-oxo-8-pyridin-3-yl-2,3-dihydro-imidazo[4,5-c]quinolin-1-yl)-phenyl]-2-methyl-propionitrile;
1-[3-Fluoro-4-(4-methanesulfonyl-piperazin-1-yl)-phenyl]-3-methyl-8-quinolin-3-yl-1,3-dihydro-imidazo[4,5-c]quinolin-2-one;
1-[3-Fluoro-4-(4-methanesulfonyl-piperazin-1-yl)-phenyl]-3-methyl-8-pyridin-3-yl-1,3-dihydro-imidazo[4,5-c]quinolin-2-one;
1-(3-Fluoro-4-piperazin-1-yl-phenyl)-3-methyl-8-quinolin-3-yl-1,3-dihydro-imidazo[4,5-c]quinolin-2-one;
1-(3-Fluoro-4-piperazin-1-yl-phenyl)-3-methyl-8-pyridin-3-yl-1,3-dihydro-imidazo[4,5-c]quinolin-2-one;
3-Methyl-1-[4-(4-methyl-piperazin-1-yl)-phenyl]-8-quinolin-3-yl-1,3-dihydro-imidazo[4,5-c]quinolin-2-one;
3-Methyl-1-[4-(4-methyl-piperazin-1-yl)-phenyl]-8-pyridin-3-yl-1,3-dihydro-imidazo[4,5-c]quinolin-2-one;
1-[2-Chloro-4-(4-methyl-piperazin-1-yl)-phenyl]-3-methyl-8-quinolin-3-yl-1,3-dihydro-imidazo[4,5-c]quinolin-2-one;
1-[2-Chloro-4-(4-methyl-piperazin-1-yl)-phenyl]-3-methyl-8-pyridin-3-yl-1,3-dihydro-imidazo[4,5-c]quinolin-2-one;
1-[3-Chloro-4-(4-methyl-piperazin-1-yl)-phenyl]-3-methyl-8-quinolin-3-yl-1,3-dihydro-imidazo[4,5-c]quinolin-2-one;
1-[3-Chloro-4-(4-methyl-piperazin-1-yl)-phenyl]-3-methyl-8-pyridin-3-yl-1,3-dihydro-imidazo[4,5-c]quinolin-2-one;
1-(4-Imidazol-1-yl-2-methyl-phenyl)-3-methyl-8-quinolin-3-yl-1,3-dihydro-imidazo[4,5-c]quinolin-2-one;
1-(4-Imidazol-1-yl-2-methyl-phenyl)-3-methyl-8-pyridin-3-yl-1,3-dihydro-imidazo[4,5-c]quinolin-2-one;
3-Methyl-1-(4-pyrazol-1-yl-phenyl)-8-quinolin-3-yl-1,3-dihydro-imidazo[4,5-c]quinolin-2-one;
3-Methyl-1-(4-pyrazol-1-yl-phenyl)-8-pyridin-3-yl-1,3-dihydro-imidazo[4,5-c]quinolin-2-one;
3-Methyl-8-quinolin-3-yl-1-(4-[1,2,4]triazol-1-yl-phenyl-1,3-dihydro-imidazo[4,5-c]quinolin-2-one;
3-Methyl-8-pyridin-3-yl-1-(4-[1,2,4]triazol-1-yl-phenyl-1,3-dihydro-imidazo[4,5-c]quinolin-2-one;
3-Methyl-1-[4-(4-methyl-piperazin-1-yl)-3-trifluoromethyl-phenyl]-8-quinolin-3-yl-1,3-dihydro-imidazo[4,5-c]quinolin-2-one;
3-Methyl-1-[4-(4-methyl-piperazin-1-yl)-3-trifluoromethyl-phenyl]-8-pyridin-3-yl-1,3-dihydro-imidazo[4,5-c]quinolin-2-one;
1-(3-Chloro-4-piperazin-1-yl-phenyl)-3-methyl-8-quinolin-3-yl-1,3-dihydro-imidazo[4,5-c]quinolin-2-one;
1-(3-Chloro-4-piperazin-1-yl-phenyl)-3-methyl-8-pyridin-3-yl-1,3-dihydro-imidazo[4,5-c]quinolin-2-one;
1-(3-Chloro-4-piperazin-1-yl-phenyl)-8-(6-methoxy-pyridin-3-yl)-3-methyl-1,3-dihydro-imidazo[4,5-c]quinolin-2-one;
1-(3-Chloro-4-piperazin-1-yl-phenyl)-8-(5-methoxy-pyridin-3-yl)-3-methyl-1,3-dihydro-imidazo[4,5-c]quinolin-2-one;
8-(6-Methoxy-pyridin-3-yl)-3-methyl-1-[4-(4-methyl-piperazin-1-yl)-3-trifluoromethyl-phenyl]-1,3-dihydro-imidazo[4,5-c]quinolin-2-one;
8-(5-Methoxy-pyridin-3-yl)-3-methyl-1-[4-(4-methyl-piperazin-1-yl)-3-trifluoromethyl-phenyl]-1,3-dihydro-imidazo[4,5-c]quinolin-2-one;
1-[2-Chloro-4-(4-methyl-piperazin-1-yl)-phenyl]-8-(6-methoxy-pyridin-3-yl)-3-methyl-1,3-dihydro-imidazo[4,5-c]quinolin-2-one;
1-[2-Chloro-4-(4-methyl-piperazin-1-yl)-phenyl]-8-(5-methoxy-pyridin-3-yl)-3-methyl-1,3-dihydro-imidazo[4,5-c]quinolin-2-one;
1-(3-Chloro-4-piperazin-1-yl-phenyl)-3-methyl-8-quinoxalin-6-yl-1,3-dihydro-imidazo[4,5-c]quinolin-2-one;
3-Methyl-1-(4-piperazin-1-yl-3-trifluoromethyl-phenyl)-8-quinolin-3-yl-1,3-dihydro-imidazo[4,5-c]quinolin-2-one;
3-Methyl-1-(4-piperazin-1-yl-3-trifluoromethyl-phenyl)-8-pyridin-3-yl-1,3-dihydro-imidazo[4,5-c]quinolin-2-one;
8-(6-Methoxy-pyridin-3-yl)-3-methyl-1-(4-piperazin-1-yl-3-trifluoromethyl-phenyl-1,3-dihydro-imidazo[4,5-c]quinolin-2-one;
8-(5-Methoxy-pyridin-3-yl)-3-methyl-1-(4-piperazin-1-yl-3-trifluoromethyl-phenyl-1,3-dihydro-imidazo[4,5-c]quinolin-2-one;
3-Methyl-1-(4-piperazin-1-yl-3-trifluoromethyl-phenyl)-8-quinoxalin-6-yl-1,3-dihydro-imidazo[4,5-c]quinolin-2-one;
1-[3-Chloro-4-(cis-3,5-dimethyl-piperazin-1-yl)-phenyl]-3-methyl-8-pyridin-3-yl-1,3-dihydro-imidazo[4,5-c]quinolin-2-one;
1-[3-Chloro-4-(cis-3,5-dimethyl-piperazin-1-yl)-phenyl]-3-methyl-8-quinolin-3-yl-1,3-dihydro-imidazo[4,5-c]quinolin-2-one;
1-[3-Chloro-4-(4-ethyl-piperazin-1-yl)-phenyl]-3-methyl-8-pyridin-3-yl-1,3-dihydro-imidazo[4,5-c]quinolin-2-one;
1-[3-Chloro-4-(4-ethyl-piperazin-1-yl)-phenyl]-3-methyl-8-quinolin-3-yl-1,3-dihydro-imidazo[4,5-c]quinolin-2-one;
1-[3-Chloro-4-(4-isopropyl-piperazin-1-yl)-phenyl]-3-methyl-8-pyridin-3-yl-1,3-dihydro-imidazo[4,5-c]quinolin-2-one;
1-[3-Chloro-4-(4-isopropyl-piperazin-1-yl)-phenyl]-3-methyl-8-quinolin-3-yl-1,3-dihydro-imidazo[4,5-c]quinolin-2-one;
1-[3-Chloro-4-(4-isopropyl-piperazin-1-yl)-phenyl]-3-methyl-8-quinolin-3-yl-1,3-dihydro-imidazo[4,5-c]quinolin-2-one;
1-[3-Chloro-4-(4-isopropyl-piperazin-1-yl)-phenyl]-3-methyl-8-quinolin-3-yl-1,3-dihydro-imidazo[4,5-c]quinolin-2-one;
1-[4-(4-Ethyl-piperazin-1-yl)-3-trifluoromethyl-phenyl]-3-methyl-8-pyridin-3-yl-1,3-dihydro-imidazo[4,5-c]quinolin-2-one;
1-[4-(4-Ethyl-piperazin-1-yl)-3-trifluoromethyl-phenyl]-3-methyl-8-quinolin-3-yl-1,3-dihydro-imidazo[4,5-c]quinolin-2-one;
1-[4-(4-Ethyl-piperazin-1-yl)-3-trifluoromethyl-phenyl]-3-methyl-8-pyridin-3-yl-1,3-dihydro-imidazo[4,5-c]quinolin-2-one;
1-[4-(4-Ethyl-piperazin-1-yl)-3-trifluoromethyl-phenyl]-3-methyl-8-quinolin-3-yl-1,3-dihydro-imidazo[4,5-c]quinolin-2-one;
3-Methyl-8-(6-piperazin-1-yl-pyridin-3-yl)-1-(3-trifluoromethyl-phenyl)-1,3-dihydro-imidazo[4,5-c]quinolin-2-one;
8-(6-Methoxy-pyridin-3-yl)-3-methyl-1-(3-trifluoromethyl-phenyl)-1,3-dihydro-imidazo[4,5-c]quinolin-2-one;
8-(6-Methoxy-pyridin-3-yl)-3-methyl-1-(3-trifluoromethyl-phenyl)-1,3-dihydro-imidazo[4,5-c]quinolin-2-one;
1-(3-Chloro-4-imidazol-1-yl-phenyl)-3-methyl-8-pyridin-3-yl-1,3-dihydro-imidazo[4,5-c]quinolin-2-one;
1-(3-Chloro-4-imidazol-1-yl-phenyl)-3-methyl-8-quinolin-3-yl-1,3-dihydro-imidazo[4,5-c]quinolin-2-one;
2-Methyl-2-[4-(3-methyl-8-quinolin-3-yl-2-thioxo-2,3-dihydro-imidazo[4,5-c]quinolin-1-yl)-phenyl]-propionitrile;
2-Methyl-2-{4-[3-methyl-8-(2-methyl-pyridin-4-yl)-2-oxo-2,3-dihydro-imidazo[4,5-c]quinolin-1-yl]-phenyl}-propionitrile;
5-{1-[4-(Cyano-dimethyl-methyl)-phenyl]-3-methyl-2-oxo-2,3-dihydro-1H-imidazo[4,5-c]quinolin-8-yl}-pyridine-2-carbonitrile;
2-[4-(4-Amino-3-methyl-2-oxo-8-quinolin-3-yl-2,3-dihydro-imidazo[4,5-c]quinolin-1-yl)-phenyl]-2-methyl-propionitrile;
1-[4-(3-Methyl-2-oxo-8-pyridin-3-yl-2,3-dihydro-imidazo[4,5-c]quinolin-1-yl)-phenyl]-cyclopropanecarbonitrile;
1-[4-(3-Methyl-2-oxo-8-quinolin-3-yl-2,3-dihydro-imidazo[4,5-c]quinolin-1-yl)-phenyl]-cyclopropanecarbonitrile;
1-{4-[8-(6-Methoxy-pyridin-3-yl)-3-methyl-2-oxo-2,3-dihydro-imidazo[4,5-c]quinolin-1-yl]-phenyl}-cyclopropanecarbonitrile;
1-[3-Chloro-4-(4-methyl-piperazin-1-yl)-phenyl]-8-(6-methoxy-pyridin-3-yl)-3-methyl-1,3-dihydro-imidazo[4,5-c]quinolin-2-one;
1-[3-Chloro-4-(4-methyl-piperazin-1-yl)-phenyl]-8-(5-methoxy-pyridin-3-yl)-3-methyl-1,3-dihydro-imidazo[4,5-c]quinolin-2-one;
1-[3-Chloro-4-(4-methyl-piperazin-1-yl)-phenyl]-3-methyl-8-quinoxalin-6-yl-1,3-dihydro-imidazo[4,5-c]quinolin-2-one;
1-(3-Chloro-4-piperazin-1-yl-phenyl)-8-(2-methoxy-pyrimidin-5-yl)-3-methyl-1,3-dihydro-imidazo[4,5-c]quinolin-2-one;
1-(3-Chloro-4-piperazin-1-yl-phenyl)-3-methyl-8-pyrimidin-5-yl-1,3-dihydro-imidazo[4,5-c]quinolin-2-one;
1-(3-Chloro-4-piperazin-1-yl-phenyl)-8-(2-methoxy-pyrimidin-5-yl)-3-methyl-1,3-dihydro-imidazo[4,5-c]quinolin-2-one;
1-(3-Chloro-4-piperazin-1-yl-phenyl)-3-methyl-8-pyrimidin-5-yl)-1,3-dihydro-imidazo[4,5-c]quinolin-2-one;
1-(3-Chloro-4-piperazin-1-yl-phenyl)-3-methyl-8-(2-methyl-pyridin-4-yl)-1,3-dihydro-imidazo[4,5-c]quinolin-2-one;
1-[3-Chloro-4-(cis-3,5-dimethyl-piperazin-1-yl)-phenyl]-8-(6-methoxy-pyridin-3-yl)-3-methyl-1,3-dihydroimidazo[4,5-c]quinolin-2-one;
1-[3-Chloro-4-(cis-3,5-dimethyl-piperazin-1-yl)-phenyl]-8-(5-methoxy-pyridin-3-yl)-3-methyl-1,3-dihydroimidazo[4,5-c]quinolin-2-one;
1-[4-(cis-3,5-Dimethyl-piperazin-1-yl)-3-trifluoromethyl-phenyl]-8-(6-methoxy-pyridin-3-yl)-3-methyl-1,3-dihydro-imidazo[4,5-c]quinolin-2-one;
1-[4-(cis-3,5-Dimethyl-piperazin-1-yl)-3-trifluoromethyl-phenyl]-8-(5-methoxy-pyridin-3-yl)-3-methyl-1,3-dihydro-imidazo[4,5-c]quinolin-2-one;
8-(2-Methoxy-pyrimidin-5-yl)-3-methyl-1-(4-piperazin-1-yl-3-trifluoromethyl-phenyl)-1,3-dihydro-imidazo[4,5-c]quinolin-2-one;
3-Methyl-1-(4-piperazin-1-yl-3-trifluoromethyl-phenyl)-8-pyrimidin-5-yl-1,3-dihydro-imidazo[4,5-c]quinolin-2-one;
5-[3-Methyl-2-oxo-1-(4-piperazin-1-yl-3-trifluoromethyl-phenyl)-2,3-dihydro-1H-imidazo[4,5-c]quinolin-8-yl]-pyridine-2-carbonitrile;
3-Methyl-8-(2-methyl-pyridin-4-yl)-1-(4-piperazin-1-yl-3-trifluoromethyl-phenyl)-1,3-dihydro-imidazo[4,5-c]quinolin-2-one;
8-(3,4-Dimethoxy-phenyl)-3-methyl-1-(4-piperazin-1-yl-3-trifluoromethyl-phenyl)-1,3-dihydro-imidazo[4,5-c]quinolin-2-one;
3-Methyl-8-pyridin-3-yl-1-(4-[1,2,4]triazol-1-yl-3-trifluoromethyl-phenyl)-1,3-dihydro-imidazo[4,5-c]quinolin-2-one;
3-Methyl-8-quinolin-3-yl-1-(4-[1,2,4]triazol-1-yl-3-trifluoromethyl-phenyl)-1,3-dihydro-imidazo[4,5-c]quinolin-2-one;
8-(6-Methoxy-pyridin-3-yl)-3-methyl-1-(4-[1,2,4]triazol-1-yl-3-trifluoromethyl-phenyl)-1,3-dihydro-imidazo[4,5-c]quinolin-2-one;
8-(5-Methoxy-pyridin-3-yl)-3-methyl-1-(4-[1,2,4]triazol-1-yl-3-trifluoromethyl-phenyl)-1,3-dihydro-imidazo[4,5-c]quinolin-2-one;
5-[3-Methyl-2-oxo-1-(4-[1,2,4]triazol-1-yl-3-trifluoromethyl-phenyl)-2,3-dihydro-1H-imidazo[4,5-c]quinolin-8-yl]-pyridine-2-carbonitrile;
8-(6-Fluoro-pyridin-3-yl)-3-methyl-1-(4-[1,2,4]triazol-1-yl-3-trifluoromethyl-phenyl)-1,3-dihydro-imidazo[4,5-c]quinolin-2-one;
8-(2,6-Dimethoxy-pyridin-3-yl)-3-methyl-1-(4-[1,2,4]triazol-1-yl-3-trifluoromethyl-phenyl)-1,3-dihydro-imidazo[4,5-c]quinolin-2-one;
3-Methyl-8-pyrimidin-5-yl-1-(4-[1,2,4]triazol-1-yl-3-trifluoromethyl-phenyl)-1,3-dihydro-imidazo[4,5-c]quinolin-2-one;
8-(2-Methoxy-pyrimidin-5-yl)-3-methyl-1-(4-[1,2,4]triazol-1-yl-3-trifluoromethyl-phenyl)-1,3-dihydro-imidazo[4,5-c]quinolin-2-one;
8-(2,4-Dimethoxy-pyrimidin-5-yl)-3-methyl-1-(4-[1,2,4]triazol-1-yl-3-trifluoromethyl-phenyl)-1,3-dihydro-imidazo[4,5-c]quinolin-2-one;
3-Methyl-1-(4-pyrazol-1-yl-3-trifluoromethyl-phenyl)-8-pyridin-3-yl-1,3-dihydro-imidazo[4,5-c]quinolin-2-one;
3-Methyl-1-(4-pyrazol-1-yl-3-trifluoromethyl-phenyl)-8-quinolin-3-yl-3-Methyl-1-(4-pyrazol-1-yl-3-trifluoromethyl-phenyl)-8-pyridin-3-yl-1,3-dihydro-imidazo[4,5-c]quinolin-2-one;
8-(6-Methoxy-pyridin-3-yl)-3-methyl-1-(4-pyrazol-1-yl-3-trifluoromethyl-phenyl)-1,3-dihydro-imidazo[4,5-c]quinolin-2-one;
8-(5-Methoxy-pyridin-3-yl)-3-methyl-1-(4-pyrazol-1-yl-3-trifluoromethyl-phenyl)-1,3-dihydro-imidazo[4,5-c]quinolin-2-one;
1-(3-Chloro-4-[1,2,4]triazol-1-yl-phenyl)-3-methyl-8-pyridin-3-yl-1,3-dihydro-imidazo[4,5-c]quinolin-2-one;
1-(3-Chloro-4-[1,2,4]triazol-1-yl-phenyl)-3-methyl-8-quinolin-3-yl-1,3-dihydro-imidazo[4,5-c]quinolin-2-one;
1-(4-Imidazol-1-yl-3-trifluoromethyl-phenyl)-3-methyl-8-pyridin-3-yl-1,3-dihydro-imidazo[4,5-c]quinolin-2-one;
1-(4-Imidazol-1-yl-3-trifluoromethyl-phenyl)-3-methyl-8-quinolin-3-yl-1,3-dihydro-imidazo[4,5-c]quinolin-2-one;
1-(4-Imidazol-1-yl-3-trifluoromethyl-phenyl)-8-(6-methoxy-pyridin-3-yl)-3-methyl-1,3-dihydro-imidazo[4,5-c]quinolin-2-one;
1-(4-Imidazol-1-yl-3-trifluoromethyl-phenyl)-8-(5-methoxy-pyridin-3-yl)-3-methyl-1,3-dihydro-imidazo[4,5-c]quinolin-2-one;
3-Methyl-8-pyridin-3-yl-1-(4-[1,2,4]triazol-1-ylmethyl-phenyl)-1,3-dihydro-imidazo[4,5-c]quinolin-2-one;
3-Methyl-8-quinolin-3-yl-1-(4-[1,2,4]triazol-1-ylmethyl-phenyl)-1,3-dihydro-imidazo[4,5-c]quinolin-2-one;
1-(4-Imidazol-1-ylmethyl-phenyl)-3-methyl-8-pyridin-3-yl-1,3-dihydro-imidazo[4,5-c]quinolin-2-one; and
1-(4-Imidazol-1-ylmethyl-phenyl)-3-methyl-8-quinolin-3-yl-1,3-dihydro-imidazo[4,5-c]quinolin-2-one;
or a tautomer thereof, or a pharmaceutically acceptable salt, or a hydrate or solvate thereof.

A very preferred compound of formula (I) of the present invention is 2-methyl-2-[4-(3-methyl-2-oxo-8-quinolin-3-yl-2,3-dihydro-imidazo[4,5-c]quinolin-1-yl)-phenyl]-propionitrile (referred to as “Compound A” herein) and its monotosylate salt. The synthesis of 2-methyl-2-[4-(3-methyl-2-oxo-8-quinolin-3-yl-2,3-dihydro-imidazo[4,5-c]quinolin-1-yl)-phenyl]-propionitrile and its monotosylate salt are for instance respectively described in WO2006/122806 as Example 7 and 152-3.

Another very preferred compound of formula (I) of the present invention is 8-(6-methoxy-pyridin-3-yl)-3-methyl-1-(4-piperazin-1-yl-3-trifluoromethyl-phenyl)-1,3-dihydro-imidazo[4,5-c]quinolin-2-one (referred to as “Compound B” herein). The synthesis of 8-(6-methoxy-pyridin-3-yl)-3-methyl-1-(4-piperazin-1-yl-3-trifluoromethyl-phenyl)-1,3-dihydro-imidazo[4,5-c]quinolin-2-one is for instance described in WO2006/122806 as Example 86.

In each embodiment described herein, the COMBINATION OF THE INVENTION comprises an amount of the compound of formula (I), or a tautomer thereof or a pharmaceutically acceptable salt, or a hydrate or solvate thereof, preferably Compound A, that is ranging from either about 1 nM to about 100 nM or about 9.5×10⁻⁸ to about 9.5×10⁻⁶ Mole/kg or about 3 to about 315 mg/subject per daily dose for the treatment of a proliferative disease, more specifically a mTOR kinase dependent proliferative disease. The COMBINATION OF THE INVENTION may include an amount of the compound of formula (I) that is ranging from about 10 to 315 mg/subject, 100 to 315 mg/subject, or 200 to 315 mg/subject per daily dose.

Accordingly, the dose amount of the compound of formula (I), or a tautomer thereof, or a pharmaceutically acceptable salt, or a hydrate or solvate thereof, preferably Compound A, in a subject in need thereof corresponds to a dose amount of about 1 nM to about 100 nM per daily dose, from about 6 nM to about 78 nM per daily dose, from about 8 nM to about 62 nM per daily dose, or from about 16 nM to about 50 nM per daily dose.

In one embodiment, the amount of the compound of formula (I), or a tautomer thereof, or a pharmaceutically acceptable salt, or a hydrate or solvate thereof, preferably Compound A, can be from about 9.5×10⁻⁸ Mole/kg to about 9.5×10⁻⁸ Mole/kg, from about 4.8×10⁻⁷ Mole/kg to about 7.4×10⁻⁶ Mole/kg, from about 7.6×10⁻⁷ Mole/kg to about 5.9×10⁻⁶ Mole/kg, or from about 1.5×10⁻⁶ Mole/kg to about 4.7×10⁻⁸ Mole/kg per daily dose.

In an alternate embodiment, the dose amount of the-compound of formula (I) or a tautomer thereof, or a pharmaceutically acceptable salt, or a hydrate or solvate thereof, preferably Compound A, for a subject in need thereof can be from about 3 mg/subject to about 315 mg/subject per daily dose, from about 15 mg/subject to about 245 mg/subject per daily dose, from about 25 mg/subject to about 195 mg/subject per daily dose, or from about 50 mg/subject to about 157 mg/subject per daily dose. The subject in need thereof is preferably a human.

In an alternate embodiment, the dose amount of the compound of formula (I) or a tautomer thereof, or a pharmaceutically acceptable salt, or a hydrate or solvate thereof, preferably Compound A, for a subject in need thereof, wherein the subject is estimated to be approximately 70 kg, can be from about 10 to 315 mg/subject, 100 to 315 mg/subject, or 200 to 315 mg/subject per daily dose.

The COMBINATION OF THE INVENTION includes compounds which target, decrease or inhibit the activity/function of the mTOR kinase through binding to the allosteric binding site of the mTORC1 complex. Such compounds will be referred to as “allosteric mTOR inhibitor compounds”. Suitable allosteric mTOR inhibitors include e.g.:
I. Rapamycin which is an immunosuppressive lactam macrolide that is produced by Streptomyces hygroscopicus.
II. Rapamycin derivatives such as:
   a. Substituted rapamycin e.g. a 40-O-substituted rapamycin e.g. as described in U.S. Pat. No. 5,258,389, WO 94/09010, WO 92/05179, U.S. Pat. No. 5,118,877, U.S. Pat. No. 5,118,678, U.S. Pat. No. 5,100,883, U.S. Pat. No. 5,151,413, U.S. Pat. No. 5,120,842, WO 93/11130, WO 94/02136, WO 94/02485 and WO 95/14023 all of which are incorporated herein by reference;
   b. a 16-O-substituted rapamycin e.g. as disclosed in WO 94/02136, WO 95/16691 and WO 95/41807, the contents of which are incorporated herein by reference;
   c. a 32-hydrogenated rapamycin e.g. as described in WO 96/41807 and U.S. Pat. No. 5,256,790, incorporated herein by reference.
   d. Preferred rapamycin derivatives are compounds of formula (II)

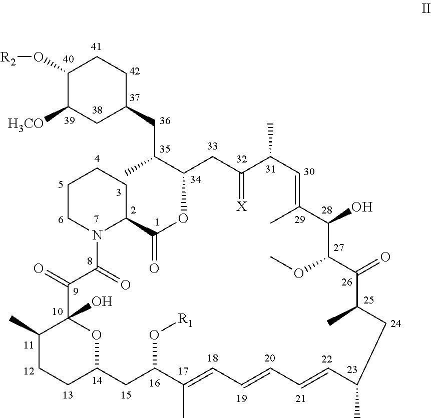

wherein

R₁ is CH₃ or C₃₋₆alkynyl,

R₂ is H or —CH₂—CH₂—OH 3-hydroxy-2-(hydroxymethyl)-2-methyl-propanoyl or tetrazolyl, and X is ═O, (H,H) or (H,OH)

provided that R₂ is other than H when X is ═O and R₁ is CH₃, or a prodrug thereof

when R₂ is —CH₂—CH₂—OH, e.g. a physiologically hydrolysable ether thereof.

Compounds of formula (II) are disclosed e.g. in WO 94/09010, WO 95/16691 or WO 96/41807, which are incorporated herein by reference. They may be prepared as disclosed or by analogy to the procedures described in these references.

Preferred compounds are 32-deoxorapamycin, 16-pent-2-ynyloxy-32-deoxorapamycin, 16-pent-2-ynyloxy-32(S)-dihydro-rapamycin, 16-pent-2-ynyloxy-32(S)-dihydro-40-O-(2-hydroxyethyl)-rapamycin and, more preferably, 40-0-(2-hydroxyethyl)-rapamycin, disclosed as Example 8 in WO 94/09010.

Particularly preferred rapamycin derivatives of formula (II) are 40-O-(2-hydroxyethyl)-rapamycin, 40-[3-hydroxy-2-(hydroxymethyl)-2-methylpropanoate]-rapamycin (also called CCI779), 40-epi-(tetrazolyl)-rapamycin (also called ABT578), 32-deoxorapamycin, 16-pent-2-ynyloxy-32(S)-dihydro rapamycin, or TAFA-93.
e. Rapamycin derivatives also include so-called rapalogs, e.g. as disclosed in WO 98/02441 and WO 01/14387, e.g. AP23573, AP23464, or AP23841.
   Rapamycin and derivatives thereof have, on the basis of observed activity, e.g. binding to macrophilin-12 (also Known as FK-506 binding protein or FKBP-12), e.g. as described in WO 94/09010, WO 95/16691 or WO 96/41807, been found to be useful e.g. as immuno-suppressant, e.g. in the treatment of acute allograft rejection.
III. Ascomycin, which is an ethyl analog of FK506.
IV. AZD08055 and OSI127, which are compounds that inhibit the kinase activity of mTOR by directly binding to the ATP-binding cleft of the enzyme

In one embodiment of the present invention, the COMBINATION OF THE INVENTION comprises at least one allosteric mTOR inhibitor compound selected from the group consisting of Sirolimus (rapamycin, AY-22989, Wyeth), Everolimus (RAD001, Novartis)), 40-[3-hydroxy-2-(hydroxymethyl)-2-methylpropanoate]-rapamycin also called Temsirolimus or CCI-779, Wyeth), Deferolimus(AP-23573/MK-8669, Ariad/Merck & Co) or a pharmaceutically acceptable salt thereof.

In the preferred embodiment of the present invention, the COMBINATION OF THE INVENTION is comprised of the allosteric mTOR inhibitor compound everolimus. Everolimus (referred to as “RAD001” T or “PKF-222-6666-NX-2” herein), has the chemical name (1R,9S,12S,15R,16E,18R,19R,21R,23S,24E,26E,28E,30S,32S, 35R)-1,18-dihydroxy-12-{(1R)-2-[(1S,3R,4R)-4-(2-hydroxyethoxy)-3-methoxycyclohexyl]-1-methylethyl}-19,30-dimethoxy-15,17,21,23,29,35-hexamethyl-11,36-dioxa-4-aza-tricyclo[30.3.1.04.9]hexatriaconta-16,24,26,28-tetraene-2,3,10,14,20-pentaone or 40-O-(2-hydroxyethyl)-rapamycin. Everolimus and analogues are described in U.S. Pat. No. 5,665,772, at column 1, line 39 to column 3, line 1, which are incorporated herein by reference hereto in its entirety. Everolimus may be prepared as disclosed or by analogy to the procedures described in this reference.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

Comprised are likewise the pharmaceutically acceptable salts thereof, the corresponding racemates, diastereoisomers, enantiomers, tautomers, as well as the corresponding crystal modifications of above disclosed compounds where present, e.g. solvates, hydrates and polymorphs, which are disclosed therein. The compounds used as active ingredients in the combinations of the invention can he prepared and administered as described in the cited documents, respectively. Also within the scope of this invention is the combination of more than two separate active ingredients as set forth above, i.e., a pharmaceutical combination within the scope of this invention could include three active ingredients or more.

It has been surprisingly discovered that unexpected synergistic interaction is achieved between compounds of formula (I) and allosteric mTOR inhibitors, particularly RAD001, when low dose amounts of the compounds of formula (I) are combined with allosteric mTOR inhibitors. The COMBINATION OF THE INVENTION may comprise a dose amount of everolimus (RAD001) comprising less than or equal to 10 mg/subject (e.g., 8 mg/subject, 5 mg/subject, 2.5 mg/subject, 1 mg/subject) per daily dose.

The COMBINATION OF THE INVENTION may comprises an dose amount of the allosteric mTOR inhibitor compound, particularly everolimus (RAD001), that is from about 0.001 nM to about 17.8 nM or from about 8.5×10⁻¹² Mole/kg to about 1.5×10⁻⁷ Mole/kg, or from about 0.00056 mg/subject to about 10 mg/subject per daily dose for the treatment of a proliferative disease.

Accordingly, the dose amount of the allosteric mTOR inhibitor compound, particularly everolimus (RAD001), administered to a subject in need thereof corresponds to a dose amount of 0.001 nM to about 17.8 nM per daily dose, from about 0.001 nM to about 10 nM per daily dose, or from about 0.001 nM to about 1 nM per daily dose. Most preferably, the dose amount of the allosteric mTOR inhibitor compound is about 0.001 nM to about 1 nM per daily dose.

In one embodiment, the dose amount of the allosteric mTOR inhibitor compound, particularly everolimus (RAD001), can be from about 8.5×10⁻¹² Mole/kg to about 1.5×10⁻⁷ Mole/kg per daily dose, from about 8.5×10⁻¹² Mole/kg to about 8.5×10⁻⁸ Mole/kg par daily dose, or from about 8.5×10⁻¹² Mole/kg to about 8.5×1.0⁻⁹ Mole/kg per daily dose. Most preferably, the dose amount of the allosteric mTOR inhibitor compound is from about 8.5×10⁻¹² Mole/kg to about 8.5×10⁻⁹ per daily dose.

In an alternate embodiment, the dose amount of the allosteric mTOR inhibitor compound, particularly everolimus (RAD001), administered to a subject in need thereof, can be from about 0.00056 mg/subject to about 10 mg/subject per daily dose, from about 0.00056 mg/subject to about 5.6 mg/subject per daily dose, or from about 0.00056 mg/subject to about 0.56 mg/subject per daily dose. Most preferably, the dose amount of the allosteric mTOR inhibitor compound is from about 0.00056 mg/subject to about 0.56 mg/ subject per daily dose. The subject in need thereof is preferably a human.

In a preferred embodiment, the COMBINATION OF THE INVENTION pertains to a pharmaceutical combination which comprises (a) Compound A or its monotosylate salt and (b) the allosteric mTOR inhibitor compound everolimus (RAD001), wherein Compound A is provided in an amount between about 1 nM to about 100 nM or about 9.5×10⁻⁸ to about 9.5×10⁻⁻⁶ Mole/kg or about 3 to about 315 mg/subject per daily dose for the treatment of an mTOR kinase dependent proliferative disease, in a further embodiment, the allosteric mTOR inhibitor compound is provided in a therapeutically effect amount from about 0.001 nM to about 17.8 nM or from about 8.5×10⁻¹² Mole/kg to about 1.5×10⁻⁷ Mole/kg, or from about 0.00056 mg/subject to about 10 mg/subject per daily dose.

In a further embodiment, the dose amount of Compound A corresponds to a dose amount of about 1 nM to about 100 nM per daily dose, from about 5 nM to about 78 nM per daily dose, from about 8 nM to about 62 nM per daily dose, or from about 16 nM to about 50 nM per daily dose.

In a further embodiment, the dose amount of Compound A can be from about 9.5×10⁻⁸ Mole/kg to about 9.5×10⁻⁻⁶ Mole/kg, from about 4.8×10⁻⁷ Mole/kg to about 7.4×10⁻⁵ Mole/kg, from about 7.6×10⁻⁷ Mole/kg to about 5.9×10⁻⁶ Mole/kg, or from about 1.5×10⁻⁶ Mole/kg to about 4.7×10⁻⁻⁶ Mole/kg per daily dose.

In an alternate embodiment, the dose amount of Compound A can be from about 3 mg/subject to about 315 mg/subject per daily dose, from about 15 mg/subject to about 245 mg/subject per daily dose, from about 25 mg/subject to about 195 mg/subject per daily dose, or from about 50 mg/subject to about 157 mg/subject per daily dose. The subject in need thereof is preferably a human.

In an alternate embodiment, the dose amount of Compound A can be from about 10 to 315 mg/subject, 100 to 315 mg/subject, or 200 to 315 mg/subject per daily dose.

In a further embodiment, the dose amount of the allosteric mTOR inhibitor compound everolimus (RAD001) administered to a subject in need thereof corresponds to a dose amount of about 0.001 nM to about about 17.8 nM per daily dose, from about 0.001 nM to about 10 nM per daily dose, or from about 0.001 nM to about 1 nM per daily dose Most preferably, the dose amount of the allosteric mTOR inhibitor compound is about 0.001 nM to about 1 nM per daily dose.

In one embodiment, the dose amount of the allosteric mTOR inhibitor compound everolimus (RAD001) can be from about 8.5×10⁻¹² Mole/kg to about 1.5×10⁻⁷ Mole/kg per daily dose, from about 8.5×10⁻¹² Mole/kg to about 8.5×10⁻⁸ Mole/kg per daily dose, or from about 8.5×10⁻¹² Mole/kg to about 8.5×10⁻⁹ Mole/kg per daily dose. Most preferably, the dose amount of the allosteric mTOR inhibitor compound is from about 8.5×10⁻¹² Mole/kg to about 8.5×10⁻⁹ per daily dose.

In an alternate embodiment, the dose amount of the allosteric mTOR inhibitor compound everolimus (RAD001) in a subject in need thereof, wherein the subject is estimated to be approximately 70 kg, can be from about 0.00056 mg/subject to about 10 mg/subject per daily dose, from about 0.00056 mg/subject to about 5.6 mg/subject per daily dose, or from about 0.00056 mg/subject to about 0.56 mg/subject per daily dose. Most preferably, the dose amount of the allosteric mTOR inhibitor compound is from about 0.00056 mg/subject to about 0.56 mg/subject per daily dose. The subject in need thereof is preferably a human.

In accordance with the present invention, the COMBINATION OF THE INVENTION may be used for the treatment of a proliferative disease, particularly an mTOR kinase dependent proliferative disease.

“mTOR kinase dependent proliferative diseases” include, but not restricted to, proliferative diseases, including cancers and other related malignancies, associated with pathological mTOR signaling cascades. A non-limiting list, of the cancers associated with pathological mTOR signaling cascades includes non small cell lung cancer, endometrial cancer, multiple myeloma, non-Hodgkin's B cell lymphoma, colorectal cancer, breast cancer, renal cell carcinoma, gastric tumors, neuroendocrine tumors, lymphomas and prostate cancer.

Preferred mTOR kinase dependent proliferative diseases are breast, glioblastomas, non small cell lung cancer, endometrial cancer, multiple myeloma, and non-Hodgkin's B cell lymphoma.

Further examples of proliferative diseases are for instance benign or malignant tumor, carcinoma of the brain, kidney, liver, adrenal gland, bladder, stomach, ovaries, colon, rectum, pancreas, lung (e.g., non small cell lung cancer), endometrial, non-Hodgkin's B-cell lymphoma, vagina or thyroid, sarcoma, glioblastomas, multiple myeloma or or gastric gastrointestinal cancer, especially colon carcinoma or colorectal adenoma or a tumor of the neck and head, an epidermal hyperproliferation, psoriasis, prostate hyperplasia, neuroendicrine, a neoplasia, a neoplasia of epithelial character, lymphomas, a mammary carcinoma or a leukemia.

In one embodiment, the present invention relates to the use of a pharmaceutical combination which comprises (a) a compound of formula (I) or a tautomer thereof, or a pharmaceutically acceptable salt, or a hydrate or solvate thereof, and (b) at least one allosteric mTOR inhibitor compound and optionally at least one pharmaceutically acceptable carrier for the treatment or prevention of a proliferative disease, particularly a mTOR kinase dependent proliferative disease, wherein the compound of formula (I) is administered to a subject in need thereof in an amount between about 1 nM to about 100 nM or about 9.5×10⁻⁸ to about 9.5×10⁻⁶ Mole/kg or about 3 to about 315 mg/subject per daily dose.

In another embodiment, the present invention relates to the use of a pharmaceutical combination which comprises (a) a compound of formula (I) or a tautomer thereof, or a pharmaceutically acceptable salt, or a hydrate or solvate thereof, and (b) at least one allosteric mTOR inhibitor compound and optionally at least one pharmaceutically acceptable carrier for the manufacture of a medicament for the treatment or prevention of a proliferative disease, particularly a mTOR kinase dependent proliferative disease, wherein the compound of formula (I) is administered to a subject in need thereof in an amount between about 1 nM to about 100 nM or about 9.5×10⁻⁸ to about 9.5×10⁻⁶ Mole/kg or about 3 to about 315 mg/subject per daily dose.

In another aspect, the present invention provides a method of treating or preventing a proliferative disease comprising administering (a) a therapeutically effective amount of a compound of formula (I) or a tautomer thereof, or a pharmaceutically acceptable salt, or a hydrate or solvate thereof, and (b) a therapeutically effective amount of at least one allosteric mTOR inhibitor compound and optionally at least one pharmaceutically acceptable carrier to a subject in need thereof, wherein the compound of formula (I) is administered in an amount, between about 1 nM to about 100 nM or about 9.5×10⁻⁸ to about 9.5×10⁶ Mole/kg or about 3 to about 315 mg/subject per daily dose.

In another aspect the present invention provides a combination comprising (a) a compound of formula (I) or a tautomer thereof, or a pharmaceutically acceptable salt, or a hydrate or solvate thereof, and (b) at least one allosteric mTOR inhibitor compound selected from the group consisting of RAD rapamycin (sirolimus) and derivatives/analogs thereof such as everolimus (or RAD001); CCI-779 and Deferolimus (AP-23573/MK-8669) or a pharmaceutically acceptable salt thereof, and optionally at least one pharmaceutically acceptable carrier, wherein the compound of formula (I) is administered to a subject in need thereof in an amount between about 1 nM to about 100 nM or about 9.5×10⁻⁸ to about 9.5×10⁻⁶ Mole/kg or about 3 to about 315 mg/subject per daily dose for simultaneous, separate or sequential use, for the treatment of proliferative diseases.

In a further aspect the present invention provides a method for improving efficacy of the treatment of a mTOR kinase dependent proliferative diseases by administering (a) a compound of formula (I) or a tautomer thereof, or a pharmaceutically acceptable salt, or a hydrate or solvate thereof, and (b) at least one allosteric mTOR inhibitor compound and optionally at least one pharmaceutically acceptable carrier to a subject in need thereof, wherein the compound of formula (I) is administered to a subject in need thereof in an amount between about 1 nM to about 100 nM or about 9.5×10⁻⁸ to about 9.5×10⁻⁶ Mole/kg or about 3 to about 315 mg/subject per daily dose.

In a further aspect, the present invention provides a pharmaceutical combination for administration to humans comprising (a) a compound of formula (I) or a tautomer thereof, or a pharmaceutically acceptable salt, or a hydrate or solvate thereof, as described above, at about 0.31% to about 31%, about 1.6% to about 24.4%, about 2.5% to about 19.4%, or about 5.0% to about 15.6% of the maximal tolerable dose (MTD) and (b) at least one allosteric mTOR inhibitor compound thereof at about 0.006% to 100%, about 0.006% to about 56.3%, about 0.006% to about 5.6% of the MTD. In a preferred embodiment, the compound of-formula (I) is Compound A which is dosed at about 30% of the MTD and the allosteric mTOR inhibitor compound is dosed at about 5.6% of the MTD. In the most preferred embodiment, the compound of formula (I) is Compound A which is dosed at about 30% of the MTD and the allosteric mTOR inhibitor compound is everolimus (RAD001) dosed at 5.6% of the MTD. The MTD corresponds to the highest dose of a medicine that can be given without unacceptable side effects. It is within the art to determine the MTD. For instance the MTD can suitably be determined in a Phase I study including a dose escalation to characterize close limiting toxicities and determination of biologically active tolerated dose level.

In one aspect of the invention, the present invention pertains to a pharmaceutical combination, such as a combined preparation or a pharmaceutical composition, comprising (a) a compound of formula (I), and (b) at least one allosteric mTOR inhibitor compound and optionally at least one pharmaceutically acceptable carrier for simultaneous, separate or sequential use, in particular for the treatment of an mammalian target of rapamycin (mTOR) kinase dependent proliferative diseases, wherein the compound of formula (I) is administered to a subject in need thereof in an amount between about 1 nM to about 100 nM or about 9.5×10⁻⁸ to about 9.5×10⁻⁶ Mole/kg or about 3 to about 315 mg/subject per daily dose.

In a preferred embodiment, the compound of formula (I) is 2-methyl-2-[4-(3-methyl-2-oxo-8-quinolin-3-yl-2,3-dihydro-imidazo[4,5-c]quinolin-1-yl)-phenyl]-propionitrile (Compound A) or its monotosylate salt.

In a further embodiment, the compound of formula (I) is 8-(6-methoxy-pyridin-3-yl)-3-methyl-1-(4-piperazin-1-yl-3-trifluoromethyl-phenyl)-1,3-dihydro-imidazo[4,5-c]quinolin-2-one (Compound B).

In a further embodiment of the present invention, the allosteric mTOR inhibitor compound is selected from the group consisting of RAD rapamycin (sirolimus) and derivatives/analogs thereof such as everolimus (or RAD001); CCI-779 and Deferolimus (AP-23573/MK-8669) or a pharmaceutically acceptable salt thereof. Particularly preferred allosteric mTOR inhibitor compounds in accordance with the present invention is everolimus.

In a preferred embodiment of the present invention, the compound of formula (I) is 2-methyl-2-[4-(3-methyl-2-oxo-8-quinolin-3-yl-2,3-dihydro-imidazo[4,5-c]quinolin-1-yl)-phenyl]-propionitrile (Compound A) or its monotosylate salt and the allosteric mTOR inhibitor compound is everolimus (RAD001).

The pharmaceutical compositions, or combination in accordance with the present invention can be tested in clinical studies. Suitable clinical studies may be, for example, open label, dose escalation studies in patients with proliferative diseases. Such studies prove in particular the synergism of the active ingredients of the COMBINATION OF THE INVENTION. The beneficial effects, e.g., synergy, on proliferative diseases may be determined directly through the results of these studies which are known as such to a person skilled in the art. Such studies may be, in particular, suitable to compare the effects of a monotherapy using the active ingredients and a COMBINATION OF THE INVENTION. Each patient may receive doses of the agent (a) either daily or intermittent. The efficacy of the treatment may be determined in such studies, e.g., after 12, 18 or 24 weeks by evaluation of symptom scores every 6 weeks.

The administration of a pharmaceutical COMBINATION OF THE INVENTION may result not only in a beneficial effect, e.g. a synergistic therapeutic effect, e.g. with regard to alleviating, delaying progression of or inhibiting the symptoms, but also in further surprising beneficial effects, e.g. fewer side-effects, an improved qualify of life or a decreased morbidity, compared with a monotherapy applying only one of the pharmaceutically active ingredients used in the COMBINATION OF THE INVENTION.

It is one objective of this invention to provide a pharmaceutical composition comprising a quantify, which may be jointly therapeutically effective at targeting or preventing mTOR kinase dependent proliferative diseases, of a COMBINATION OF THE INVENTION. In this composition, agent (a) and agent (b) may be administered together, one after the other or separately in one combined unit dosage form or in two separate unit dosage forms. The unit dosage form may also be a fixed combination.

The pharmaceutical compositions for separate administration of combination partner (a) and combination partner (b) or for the administration in a fixed combination, i.e. a single galenical composition comprising at least two combination partners (a) and (b), according to the invention may be prepared in a manner known per se and are those suitable for enteral, such as oral or rectal, and parenteral administration to mammals (warm-blooded animals), including humans, comprising an amount of at least one pharmacologically active combination partner alone, e.g. as indicated above, or in combination with one or more pharmaceutically acceptable carriers or diluents, especially suitable for enteral or parenteral application.

Pharmaceutical preparations for the combination therapy for enteral or parenteral administration are, for example, those in unit dosage forms, such as sugar-coated tablets, tablets, capsules or suppositories, or ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount may be reached by administration of a plurality of dosage units.

In preparing the compositions for oral dosage form, any typical pharmaceutical acceptable carriers or excipients may be added to the components of the composition, which can be solid or liquid. Solid form preparations comprise, for example, powders, capsules and tablets. Examples of pharmaceutically acceptable carrier include water, glycols, oils, alcohols, flavoring agents, preservatives, coloring agents; or carriers such as starches, sugars, microcristalline cellulose, diluents, granulating agents, lubricants, binders, disintegrating agents and the like in the case of oral solid preparations, with the solid oral preparations being preferred over the liquid preparations. Because of their ease of administration, tablets and capsules represent the most advantageous oral dosage unit form in which case solid pharmaceutical carriers are obviously employed. Pharmaceutical compositions comprising a catalytic PI3K/mTOR inhibitor compound of formula (I) and most preferably Compound A, in association with at least one pharmaceutically acceptable carrier may be manufactured in a conventional manner by mixing with a pharmaceutically acceptable carrier.

Liquid form preparations comprise solutions, suspensions, and emulsions. Liquid compositions can be formulated in solution by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, and other suspending agents known to the pharmaceutical formulation art.

In particular, any amount of each of the combination partner of the COMBINATION OF THE INVENTION may be administered simultaneously or sequentially and in any order, and the components may be administered separately or as a fixed combination. For example, the method of preventing or treating a mTOR kinase dependent proliferative disease according to the invention may comprise (I) administration of the combination partner (a) in free or pharmaceutically acceptable salt form and (ii) administration of a combination partner (b) in free or pharmaceutically acceptable salt form, simultaneously or sequentially in any order, in jointly amounts, preferably in synergistically effective amounts, e.g. in daily or intermittently dosages corresponding to the amounts described herein. The individual combination partners of the COMBINATION OF THE INVENTION may be administered separately at different times during the course of therapy or concurrently in divided or single combination forms. Furthermore, the term “administering” also encompasses the use of a pro-drug of a combination partner that convert in vivo to the combination partner as such. The instant invention is therefore to be understood as embracing all such regimens of simultaneous or alternating treatment and the term “administering” is to be interpreted accordingly. Further the term “daily dose” encompasses the amount of the individual combination partners of the COMBINATION OF THE INVENTION, as separately at different times during the course of therapy or concurrently in divided or single dose unit forms, administered that is equal to or equivalent to the amount specified during any 24-hour period.

The effective dosage of each of the combination partners employed in the COMBINATION OF THE INVENTION may vary depending on the particular compound or pharmaceutical composition employed, the mode of administration, the condition being treated, the severity of the condition being treated. Thus, the dosage regimen of the COMBINATION OF THE INVENTION is selected in accordance with a variety of factors including the route of administration and the renal and hepatic function of the patient. A clinician or physician of ordinary skill can readily determine and prescribe the effective amount of the single active ingredients required to alleviate, counter or arrest the progress of the condition. Optimal precision in achieving concentration of the active ingredients within the range that yields efficacy without toxicity requires a regimen based on the kinetics of the active ingredients' availability to target sites.

In another embodiment, the invention pertains to a kit of parts comprising a pharmaceutical composition comprising (a) a compound of formula (I), and (b) at least one allosteric mTOR inhibitor compound together with instructions how to administer that pharmaceutical composition, wherein the compound of formula (I) is administered to a subject in need thereof in an amount between about 1 nM to about 100 nM or about 9.5×10⁻⁸ to about 9.5×10⁻⁶ Mole/kg or about 3 to about 315 mg/subject per daily dose. These instructions will explain in detail the dosing regimen how the combination is to be administered.

In one embodiment, the invention pertains to a kit of parts comprising a pharmaceutical composition comprising Compound A. and at least one allosteric mTOR inhibitor compound, preferably everolimus (RAD001). together with instructions how to administer that pharmaceutical composition, wherein Compound A is administered to a subject in need thereof in an amount between about 1 nM to about 100 nM or about 9.5×10⁻⁸ to about 9.5×10⁻⁶ Mole/kg or about 3 to about 315 mg/subject per daily dose. These instructions will explain in detail the dosing regimen how the combination is to be administered.

The present invention further provides a commercial package comprising as active ingredients COMBINATION OF THE INVENTION, together with instructions for simultaneous, separate or sequential use thereof in the delay of progression or treatment of a mTOR kinase dependent proliferative disease.

The following Examples illustrate the invention described above; they are not, however, intended to limit the scope of the invention in any way. The beneficial effects of the pharmaceutical combination of the present invention can also be determined by other test models known as such to the person skilled in the pertinent art.

### EXAMPLE 1

### Material and Methods

The cell lines used in this study were purchased from American Type Cell Collection, including non small cell lung cancer cell line NCI-H23 (which carries both KRAS and LKB1 mutations), endometrial tumor cell lines MFE 296 (which carries both PIK3CA and PTEN mutations) and AN 3CA (which carries both FGFR2 and PTEN mutations). Multiple Myeloma cell lines KMS 11 (which carries FGFR3 mutations) and RPMI 8226, Non-Hodgkin's B cell lymphoma line GA-10. All the cell lines were cultured at 37° C. in a 5% CO2 incubator in RPMI 1640 (ATCC #30-2001) media complemented with 10% fetal bovine serum, 2 mmol/L glutamine and 1% sodium pyruvate.

Celt Proliferation Assay: Cell viability was determined by measuring cellular ATP content using the CellTiter-Glo® Luminescent Cell Viability Assay (Promega #G7573) according to manufacturer's protocol. Briefly, 1500-50000 cells were plated on either 384 or 96 well plates in 30 μl (384 well) or 100 μl (96 well) growth media, cells were: allowed to attach overnight and followed by 72 hrs of incubation with various concentration of drugs or drug combinations (10 μl per well in 384 well plates), at the end of the drug treatment, 30 μl of the CellTiter-Glo regent were added to each well (384 well plates) to lyse the cell, and luminescence signals were recorded on a Envision plate reader.

Automated imaging assay (or High-Content assay) for pS6 S240/244 and p4EBP1 T37/46: 2-4×10³ cells were seeded in clear-bottom 384-well black plates (Greiner #781091) in 30 μl per well growth media 24 hours prior to treatment Compounds were added to the cell in 10 μl growth media and incubated overnight; the cells were then fixed by addition of 10 μl per well Mirsky's fixative (National Diagnostics #HS-102) for 1 hour, washed seven times with 30 μl/well TBS buffer using a BioTek plate washer, and blocked with 100 μl/well blocking buffer (TBS with 0.1% BSA and 0.1% Triton X-100). The anti-Ser 240/244 -RPS6 antibody (CST #4838, 1:150 dilution) or anti-Thr 37/46-p4EBP1 antibody (CST #2855, 1:150 dilution) were then incubated overnight at 4° C. After seven times wash with TBS, the cells were stained with Cy5-conjugated goat-anti-rabbit IgG 2^{nd} antibody (Millipore #AP187S, 1:150 dilution) and DNA staining dye Neediest 33342 for 1.5 hours. The phospho-S6 and phospho-4EBP1 signals were-imaged using InCell 1000 Analyzer (BN_staining_{—}10×protocol) at 10× magnification, 3 fields per well after seven times wash with TBS. For Hoechst 33342 signal; the excitation and emission wave lengths are 360 nm (D360_{—}40× filter) and 460 nM (HQ460_{—}40M filter), respectively, and for Cy5, 620 nm (HQ620_{—}60× filter) for both the excitation and emission wave lengths. Analysis of the images was done using the InCell Investigator software.

Method for calculating the effect of the Combination: To evaluate the everolimus and Compound A combination effect in a non-bias way and to identify synergistic effect at all possible concentrations, the combination studies were conducted with a “dose matrix”, where a combination is tested in all possible permutations of serially-diluted everolimus and Compound A single agent doses, in all combination assays, compounds were applied simultaneously. Single agent dose responding curves, IC₅₀, IC₉₀, and the Synergy are all analyzed using Chalice software (CombinatoRx, Cambridge, Mass.). Synergy was calculated by comparing a combination's response to those of its single agents, against the drug-with-itself dose-additive reference model. Deviations from dose additivity can be assessed visually on an Isobologram or numerically With a Combination Index. Excess inhibition compare to additivity can also be plotted as a full dose-matrix chart to capture where the synergies occur. To quantify the overall strength of combination effects, a volume score V_{HSA}=Σ_{XY}Inf_{X}Inf_{Y}(I_{data}−I_{HSA}) is also calculated between the data and the highest-single-agent surface, normalized for single agent dilution factors f_{X},f_{Y}[ref].

### Results:

### A. Effect of Combination in NCI-H23 Human Non-Small Cell Lung Cancer (NSCLC) Cell Model

p4EBP1 signal: The effect of single agent and concomitant everolimus/Compound A treatment on the p4ESP1 signal was evaluated using the high content p4EBP1 T37/46 assay described above. The cells were plated at 3000 cells per well in 384 well plates in quadruplicates, and treated with compound for 18 hrs before the measurement (FIGS. 1-2). In this “dose matrix” study, everolimus was subjected to a 5 dose 4× serial dilution with the high: dose at 1.2 μM and the low dose at about 5 nM, and Compound A was subjected to a 9 dose 2× serial dilution with high dose at 1.2 μM and low dose at about 5 nM Compound A alone caused a concentration-dependent reduction of p4EBP1 signal (IC₅₀=10 nM, and IC₉₀=80 nM), and the signal reduction plateaued at concentrations of 167 nM and above where complete inhibition is apparently achieved; everolimus as a single agent, only exerted a very marginal effect on p4EBP1 signals at all concentrations tested (5 nM-1.2 μM, approximately 30% signal reduction). This is consistent with the previous reports that phosphorylation of the 4EBP1 T37/46 residues, which plays a key role in regulating cap-dependent translation, can only be modulated by catalytic mTOR inhibitors, but not allosteric inhibitors like everolimus. Concomitant everolimus/Compound A treatment has markedly enhanced the inhibitor/effect compared to both everolimus at all doses (5 nM-1.2 μM, or 0.042-10.08 nMolar/kg, or 2.82-676.44 mg/person) and sub-optimal doses of Compound A (5 nM-78 nM, or 0.47-7.44 nMolar/kg, or 15.68-244.62 mg/person), at higher Compound A concentrations (156 nM-1.2 μM, or 14.88-114.50 nMolar/kg, or 489.24-3763.44 mg/person), the combination did not exhibit any additional benefit compare to Compound A single agent treatments when the maximum effect has been reached. Based on this pattern, the synergy effect observed can be classified as “dose sparing” for Compound A rather than “overall effect boosting”: as little as 5 nM of everolimus can shift the IC₉₀ for Compound A from 80 nM to 5 nM, achieving a 16 fold reduction.

pS6 S240/244 signal: The effect of single agent and concomitant everolimus/Compound A treatment on the pS6 signal was evaluated using the high content pS6 S240/244 assay described above. The experiment setup is identical to the p4EBP1 assay used for the NCI-H23 cell model (FIGS. 3-4). And the same “dose matrix” (everolimus: 5 dose, 4×, 1.2 μM to 5 nM, Compound. A: 9 dose, 2×, 1.2 μM to 5 nM) was applied. Unlike the inhibition on p4EBP1, both Compound A and everolimus as single agents displaced very potent inhibitory effect on pS6 signal: IC₅₀ for Compound A is 5 nM and IC₉₀ about 20 nM, while everolimus's IC₅₀ is <5 nM and IC₉₀ about 10 nM. Concomitant everolimus/Compound A treatments did not result in enhanced inhibition especially compare to everolimus single agent treatment.

Cell Proliferation: The effect of single agent and concomitant everolimus/Compound A treatment on cell proliferation was evaluated using the cell titer glow (CTG) assay described above. The experiment setup is identical to the p4EBP1 high content assay as used for the NCI-H23 cell model, (FIG. 5). And the same “doss matrix” (everolimus; 5 dose, 4×, 1.2 μM to 5 nM, Compound A: 9 dose, 2×, 1.2 μM to 5 nM) was applied. Compound A alone caused a concentration-dependent inhibition of cell growth with IC₅₀=78 nM, and Amax, the maximum fraction of inhibition=0.7 (70% growth inhibition compare to DMSO control); everolimus only displaced a minor growth inhibitory effect on cell proliferation as a single agent, never achieved an IC₅₀, and Aₘₐₓ=0.3. Concomitant everolimus/Compound A treatment has dramatically enhanced, the inhibitory effect compared to both everolimus at all doses (5 nM-1.2 μM or 0.042-10.08 nMolar/kg, or 2.82-676.44 mg/person) and sub-optimal doses of Compound A (5 nM-78 nM, or 0.47-7.44 nMolar/kg, or 15.68-244.62 mg/person), at higher Compound A concentrations (156 nM-1.2 μM, or 14.88-114.50 nMolar/kg, or 489.24-3763.44 mg/person), the combination did not exhibit any additional benefit compare to Compound A single agent treatments. This pattern is highly similar to the synergy pattern of p4EBP1 inhibition: the areas of combination benefit almost overlap with each other, indicating that the synergistic inhibition of p4EBP1 is at least part (if not all) of the underlying mechanism for the observed synergy on growth inhibition. And as discussed above, this combination benefit should be categorized as “dose sparing” for Compound A rather than “overall effect boosting”; as little as 5 nM of everolimus can achieve an 8-fold reduction on IC₅₀ (from 80 nM to 10 nM), while the combination effects at all doses never exceeded the single dose effect of Compound A at high concentration (1.2 μM).

To further investigate the effect of concomitant everolimus/Compound A treatment on cell proliferation at even lower everolimus and Compound A concentrations (5 nM everolimus and Compound A combination already appeared to be highly synergistic as summarized in the cell proliferation results above for the NCI-H23 cancer cell model). Another experiment was conducted to evaluate the combination effect using cell titer glow (CTG) assay, this time, cells were plated in 384 well plates at 1000 cells/well in triplicates, and treated with compound for 72 hours before the measurement (FIG. 6). in this extended “dose matrix” study, everolimus was subjected to a 11 dose 4× serial dilution with the highest dose at 1 uM and the low dose at about 1 pM, and Compound A was subjected to a 9 dose 4× serial dilution with high dose at 1 μM and low dose at about 16 pM. The single agent activities for both Compound A and everolimus are consistent with what has been observed in the cell proliferation results above for the NCI-H23 cancer cell model: Compound A alone caused a concentration-dependent inhibition of cell growth (IC₅₀=80 nM, Aₘₐₓ=0.7), and everolimus had only a minor growth inhibitory effect as a single agent (IC₅₀>1 μM, and Aₘₐₓ=0.3); however, addition of everolimus to low dose Compound A (1-62 nM, or 0.095-5.91 nMolar/kg, or 3.14-194.44 mg/person) was able to significantly boost Compound A's antiproliferative effect to an extend that is comparable to high dose Compound A (250 nM-1 μM or 23.85-95.41 nMolar/kg, or 784.05-3136.20 mg/person), combination at sub-nanomolar amount (16 pM-250 pM) Compound A or higher dose (250-1 μM) of Compound A did not yield any benefit compared to Compound A single agent. The quantify of everolimus needed to achieve the Compound A dose sparing is also astonishingly low, as little as 1 pM, or 0.0000084 nMolar/kg, or 0.00056 mg/person of everolimus appears to be comparable to μM quantity of everolimus in term of achieving similar scale of synergy with Compound A. Suggesting that practically, trace amount of everolimus (sub nanomolar or even picomolar, 1 pM to 1 nM, or 0.0000084 to 0.0084 nMolar/kg, or 0.00056 to 0.56 mg/person) can be added to sub-optimal amount of Compound A (about 1-100 nM, or 0.095-9.54 nMolar/kg, or 3.14-313.62 mg/person) to potentiate Compound A effect on complete inhibition of mTORC1 activity and subsequently, better inhibition on growth inhibition.

### B. Effect of Combination in MPE 296 Human Endometrial Cancer Cell Model

p4EBP1 signal. The effect of single agent and concomitant everolimus/Compound A treatment on the p4EBP1 signal was evaluated using the high content p4EBP1 T37/46 assay described above. The cells were plated at 4000 cells/well in 384 well plates in duplicates, and treated with compound for 18 hrs before the measurement (FIG. 7). Similar to the “dose matrix” study performed for the HCI-H23 cancer cell model, in this “dose matrix” study, everolimus was subjected to a 11 dose 4× serial dilution with the high dose at 500 nM and the low dose at about 0.25 pM, and Compound A was subjected to a 9 dose 4× serial dilution with high dose at 1 μM and low dose at about 16 pM. Compound A alone caused a concentration-dependent reduction of p4EBP1 signal (IC₅₀=16 nM, and IC90 o about 100 nM), and reached complete inhibition at concentration >250 μM; everolimus as a single agent, only exerted a very marginal effect on p4EBP1 signals at all concentrations tested (0.5 pM-500 nM, approximately 20% signal reduction). Concomitant everolimus/Compound A treatment has markedly enhanced the inhibitory effect, compared to both everolimus at all doses (0.5 pM-500 nM, or 0.0000042-4.2 nMolar/kg, or 0.00028-281.84 mg/person) and sub-optimal doses of Compound A (16 pM-62 nM, or 0.0015-5.91 nMolar/kg, or 0.050-194.44 mg/person), the presence of trace amount of everolimus was able, to shift the IC₅₀ for Compound A from 16 nM to 0.24 nM and IC90 from about 100 nM to 4 nM. At higher Compound A concentrations (250 nM-1 μM, or 23.85-95.41 nMolar/kg, or 784.05-3136.20 mg/person), the combination did not exhibit any additional benefit compare to Compound A single agent treatments. This pattern, which is in total agreement with what has been observed in the NCI-H23 cells described above suggesting that the synergy effect observed can be categorized as “dose sparing” for Compound A without any “overall effect boosting”. Low dose combination of everolimus and Compound A can be used as a highly efficacious mTOR1 inhibition reagent.

Cell Proliferation: The effect of single agent and concomitant everolimus/Compound A treatment on cell proliferation was evaluated using the cell titer glow (CTG) assay describe above. The experiment setup is identical to the p4EBP1 assay as described above for the MFE296 cancer cell model. (FIG. 8). And the same “dose matrix” (everolimus: 11 dose, 4', 500 nM to 0.5 pM, Compound A; 9 dose, 4×, 1 μM to 16 nM) was applied. Compound A alone caused a concentration-dependent inhibition of cell growth (IC₅₀=78 nM, Aₘₐₓ=0.79), everolimus as a single agent is slightly less efficacious (Aₘₐₓ=0.6) in this cell line, however, extremely potent (IC₅₀<0.25 pM). Concomitant everolimus/Compound A treatment has markedly enhanced the inhibitory effect compared to both everolimus at all doses (0.5 pM-500 nM, or 0.0000042-4.2 nMolar/kg, or 0.00028-281.84 mg/person) and sub-optimal doses of Compound A (16 pM-62 nM, or 0.0015-5.91 nMolar/kg, or 0.050-194.44 mg/person), at higher Compound A concentrations (250 nM-1 μM, or 23.85-95.41 nMolar/kg, or 784.05-3136.20 mg/person), the combination did not exhibit any additional benefit compare to Compound A single agent treatments. The synergy pattern here again, suggested that trace amount (picomolar) of everolimus was able to significantly reduce the full effective dose of Compound A (from 250 nM to close to sub-nanomolar range).

### C. Effect of Combination in AN3 CA Human Endometrial Cancer Cell Model

Cell Proliferation: The effect of single agent and concomitant everolimus/Compound A treatment on cell proliferation was evaluated using the cell titer glow (GTG) assay described above. The cells were plated at 1500 cells/well in 384 well plates in quadruplicates, and treated with compound for 72 hrs before the measurement (FIG. 9). The following “dose matrix”, everolimus: 11 dose, 4×, 500 nM to 0.5 pM, Compound A: 9 dose, 4×, 1 μM to 16 nM, was applied. Compound A alone caused a concentration-dependent inhibition of cell growth (IC₅₀=5 nM, Aₘₐₓ=0.69); everolimus as a single agent is not very efficacious (Aₘₐₓ=0.3). Concomitant everolimus/Compound A treatment has dramatically enhanced the inhibitory effect compared to both everolimus at all doses (0.5 pM-500 nM, or 0.0000042-4.2 nMolar/kg, or 0.00028-281.84 mg/person) and sub-optimal doses of Compound A (16 pM-16 nM, or 0.0015-1.53 nMolar/kg, or 0.050-50.17 mg/person), at higher Compound A concentrations (64 nM-1 μM, or 5.92-95.42 nMolar/kg, or 194.44-3136.20 mg/person), the combination did not exhibit any additional benefit compare to Compound A single agent treatments. The synergy pattern here again, suggested a dose sparing model that trace amount of (picomolar) of everolimus was able to significantly reduce the full effective dose amount of Compound A (from >64 nM to close to nanomolar or sub-nanomolar range).

### D. Effect of Combination in GA-10 Human Non-Hodgkin's Lymphoma Cancer Cell Model

Cell Proliferation: The effect of single agent and concomitant everolimus/Compound A treatment on cell proliferation was evaluated using the cell titer glow (CTG) assay described above. The cells were plated at 50000 cells/well in 96 well plates in triplicate, and treated with compound for 72 hrs prior to the measurement (FIG. 10). The following “dose matrix”, everolimus: 8 dose, 3×, 500 nM to 0.23 nM, Compound A: 8 dose, 2×, 1 μM to 8 nM, was applied. Compound A alone caused a concentration-dependent inhibition of cell growth, and at high concentration, eliminated almost all surviving cells (IC₅₀ about 25 nM, IC₉₀=250 nM, Aₘₐₓ=1); everolimus as a single agent is not very efficacious (Aₘₐₓ=0.3). Concomitant everolimus/Compound A treatment has dramatically enhanced the inhibitory effect compared to both everolimus at all doses (0.23 nm-500 nM, or 0.0019-4.2 nMolar/kg, or 0.13-281.84 mg/person) and sub-optimal doses of Compound A (8 nM-62 nM, or 0.76-5.92 nMolar/kg, or 25.09-194.44 mg/person), shifted the IC₉₀ for Compound A from about 300 nM to 16 nM. At higher Compound A concentrations (250 nM-1 μM, or 23.85-95.41 nMolar/kg, or 784.05-3136.20 mg/person), the combination did not exhibit any additional benefit compare to Compound A single agent treatments. The synergy pattern here again, suggested a dose sparing model that trace amount of (sub-nanomolar) of everolimus was able to significantly reduce the full effective dose amount of Compound A (from >250 nM to 16-62 nM).

### E. Effect of Combination in KMS-11 Human Multiple Myeloma Cancer Cell Model

Cell Proliferation: The effect of single agent and concomitant everolimus/Compound A treatment on cell proliferation was evaluated using the cell titer glow (CTG) assay described above. The cells were plated, at 50000 cells/well in 96 well plates in triplicates, and treated with compound for 72 hrs prior to the measurement (FIG. 11). The following “dose matrix”, everolimus; 8 dose, 3×, 500 nM to 0.23 nM, Compound A: 8 dose, 2×, 1 μM to 8 nM, was applied. Compound A alone caused a concentration-dependent inhibition of cell growth, and at high concentration, (IC₅₀ about 80 nM, Aₘₐₓ=0.7); everolimus as a single agent is not very efficacious (Aₘₐₓ=0.28). Concomitant everolimus/Compound A treatment has dramatically enhanced the inhibitory effect compared to both everolimus at all doses (0.23 nM-500 nM, or 0.0019-4.2 nMolar/kg, or 0.13-281.84 mg/person) and sub-optimal doses of Compound A (8 nM-62 nM, or 0.76-5.92 nMolar/kg, or 25.09-194.44 mg/person), shifted the IC₅₀ for Compound A from about 80 nM to 16 nM. At higher Compound A concentrations (125 nM-1 uM, or 11.93-95.42 nMolar/kg, or 392.03-3136.20 mg/person), the combination did not exhibit any additional benefit compare to Compound A single agent treatments. The synergy pattern here again, suggested a dose sparing model that trace amount of (sub-nanomolar) of everolimus was able to significantly reduce the full effective dose amount of Compound A (from >125 nM to 16-82 nM).

### F. Effect of Combination in RPMI 8226 Human Multiple Myeloma Cancer Cell Model

Cell Proliferation: The effect of single agent and concomitant everolimus/Compound A treatment on cell proliferation was evaluated using the cell titer glow (CTG) assay described above. The cells were plated at 50000 cells/well in 96 well plates in triplicates, and treated with compound for 72 hrs prior to the measurement (FIG. 12). The following “dose matrix”, everolimus: 8 dose, 3×, 500 nM to 0.23 nM, Compound A: 8 dose, 2×, 1 μM to 8 nM, was applied. Compound A alone caused a concentration-dependent inhibition of cell growth, and at high concentration, (IC₅₀ about 125 nM, Aₘₐₓ=0.7); everolimus as a single agent is not very efficacious (Aₘₐₓ=0.15). Concomitant everolimus/Compound A treatment has dramatically enhanced the inhibitory effect compared to both everolimus at all doses (0.23 nM-500 nM, or 0.0019-4.2 nMolar/kg, or 0.13-281.84 mg/person) and sub-optimal doses of Compound A (8 nM-62 nM, or 0.76-5.92 nMolar/kg, or 25:09-194.44 mg/person), shifted the IC₅₀ for Compound A from about 125 nM to 16 nM. At higher Compound A concentrations (125 nM-1 μM, or 11.93-95.42 nMolar/kg, or 392.03-3136.20 mg/subject), the combination did not exhibit any additional benefit compare to Compound A single agent treatments. The synergy pattern here again, suggested a dose sparing model that trace amount of (sub-nanomolar) of everolimus was able to significantly reduce the full effective dose amount of Compound. A (from >125 nM to 16-62 nM).

### SUMMARY AND DISCUSSION

The antiproliferative effect of everolimus and Compound A combinations were evaluated in six cell lines from various tissue lineages that carry different genetic alterations, strong synergy was found in all cell tines tested with a similar pattern: everolimus was able to enhance the potency of Compound A by 5-100 fold depends on the cell types and the absolute level of enhancement depends on the differences between the maximum efficacies of Compound A and everolimus. Only trace amounts of everolimus (pM-nM) is needed to synergize with low dose Compound A (nM). Evaluation of the combination effect oh p4EBP1 reduction, a key readout for mTORC1 function identified overlapping area of synergy with the antiproliferative analysis, suggesting that the combination benefit is at least partially contributed by the synergistic inhibition of eIF4E regulated cap dependent translation pathway. Clinically, combining a fixed low dose of everolimus with an optimal low dose Compound A to achieve complete inhibition of mTORC1 (super mTORC1 inhibitor) should be a very attractive option. Compared to using Compound A as a single agent at high dose to achieve the same goal, such combination will provide the same extent of mTORC1 inhibition while avoiding the potential drug bio-availability issue of Compound A and possible off-target toxicity that might be associated with Compound A high dose.

### Example 2

The effect of single agent and concomitant everolimus/Compound A treatment on cell proliferation was evaluated in SK-BR3 Human Breast Cancer cells using the cell titer glow (CTG) assay described above. The cells were plated at 2000 cells/well in 96 well plates in triplicates, and treated with compound for 72 hrs prior to the measurement. The following dose matrix, everolimus: 8 dose, 2×, 200 nM to 15 nM, Compound A: 8 dose, 2×, 2 μM to 15 nM, was applied. Compound A alone caused a concentration-dependent inhibition of cell growth, and at high concentration. (IC₅₀ about 31 nM, Aₘₐₓ=0.67); everolimus as a single agent is also efficacious (Aₘₐₓ=0.50, IC₅₀<15 nM). Concomitant everolimus/Compound A treatment enhanced the inhibitory effect compared to both everolimus at all doses (15 nM-2 μM) and sub-optimal doses of Compound A (15 nM-60 nM), shifted the IC₅₀ for Compound A from about 31 nM to as low as <15 nM. At higher Compound A concentrations (120 nM-2 μM), the combination did not exhibit any additional benefit compare to Compound A single agent treatments. The synergy pattern here again, suggests a dose sparing model that low dose everolimus is able to significantly reduce the full effective dose range of Compound A (from 120 nM to <15 nM).

### Example 3

The effect of single agent and concomitant everolimus/Compound A treatment on cell proliferation was evaluated in MDA-MB-361 Human Breast Cancer cells using the cell titer glow (CTG) assay described above. The cells were plated at 2000 cells/well in 95 well plates in triplicates, and treated with compound for 72 hrs prior to the measurement. The following “dose matrix”, everolimus: 8 dose, 2×, 2000 nM to 15 nM, Compound A: 8 dose, 2×, 2 μM to 15 nM, was applied. Compound A alone caused a concentration-dependent inhibition of cell growth, and at high concentration, (IC₅₀ about 60 nM, Aₘₐₓ=0.81); everolimus as a single agent is not highly efficacious (Aₘₐₓ<0.50). Concomitant everolimus/Compound A treatment enhanced the inhibitory effect compared to both everolimus at all doses (15 nM-2 μM) and sub-optimal doses of Compound A (15 nM-60 nM), shifted the IC₅₀ for Compound A from about 60 nM to as low as <15 nM. At higher Compound A concentrations (120 nM-2 μM), the combination did not exhibit any additional benefit compare to Compound A single agent treatments. The synergy pattern here again, suggested a dose sparing model that low dose everolimus was able to significantly reduce the full effective dose range of Compound A (from 120 nM to <15 nM).

1. A pharmaceutical combination comprising:
a) a compound of formula (I) 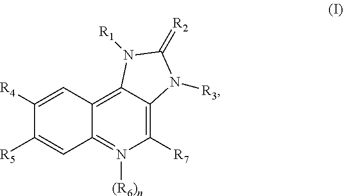
wherein
R₁ is naphthyl or phenyl wherein said phenyl is substituted by one or two substituents independently selected from the group consisting of Halogen; lower alkyl unsubstituted or substituted by halogen, cyano, imidazolyl or triazolyl; cycloalkyl; amino substituted by one or two substituents independently selected from the group consisting of lower alkyl, lower alkyl sulfonyl, lower alkoxy and lower alkoxy lower alkylamino; piperazinyl unsubstituted or substituted by one or two substituents independently selected from the group consisting of lower alkyl and lower alkyl sulfonyl; 2-oxo-pyrrolidinyl; lower alkoxy lower alkyl; imidazolyl; pyrazolyl; and triazolyl;
R₂ is O or S;
R₃ is lower alkyl;
R₄ is pyridyl unsubstituted or substituted by halogen, cyano, lower alkyl, lower alkoxy or piperazinyl unsubstituted or substituted by lower alkyl; pyrimidinyl unsubstituted or substituted by lower alkoxy; quinolinyl unsubstituted or substituted by halogen; quinoxalinyl; or phenyl substituted with alkoxy
R₅ is hydrogen or halogen;
n is 0 or 1;
R₆ is oxido;
with the proviso that if n=1, the N-atom bearing the radical R₆ has a positive charge;
R₇ is hydrogen or amino;
or a tautomer thereof, or a pharmaceutically acceptable salt, or a hydrate or solvate thereof, and
b) at least one allosteric mTOR inhibitor compound, and optionally at least one pharmaceutically acceptable carrier, for use in the treatment of a proliferative disease, wherein the compound of formula (I) is administered to a subject in need thereof in an amount between about 1 nM to about 100 nM or about 9.5×10⁻⁸ to about 9.5×10⁻⁶ Mole/kg or about 3 to about 315 mg/subject per daily dose.

2. A pharmaceutical combination of claim 1 wherein the compound of formula (I) is 2-methyl-2-[4-(3-methyl-2-oxo-8-quinolin-3-yl-2,3-dihydro-imidazo[4,5-c]quinolin-1-yl)-phenyl]-propionitrile (Compound A) and its monotosylate salt.

3. A pharmaceutical combination of claim 1 wherein the compound of formula (I) is 8-(6-methoxy-pyridin-3-yl)-3-methyl-1-(4-piperazin-1-yl-3-trifluoromethyl-phenyl)-1,3-dihydro-imidazo[4,5-c]quinolin-2-one (Compound B).

4. A pharmaceutical combination according to claim 1 wherein the allosteric mTOR inhibitor compound is selected from RAD rapamycin (sirolimus) and derivatives/analogs thereof such as everolimus (or RAD001); CCI-779 and Deferolimus (AP-23573/MK-8669).

5. A pharmaceutical combination of claim 4, wherein the allosteric mTOR inhibitor compound is everolimus (RAD001) which is administered to a subject in need thereof in an amount between administered from about 0.001 nM to about 17.8 nM or from about 8.5×10⁻¹² Mole/kg to about 1.5×10⁻⁷ Mole/kg, or from about 0.00056 mg/subject to about 10 mg/subject per daily dose.

6. A pharmaceutical combination of claim 1, wherein the proliferative disease is an mTOR kinase dependent proliferative disease.

7. A pharmaceutical combination of claim 1, wherein the proliferative disease is a benign or malignant tumor, carcinoma of the breast, brain, kidney, liver, adrenal gland, bladder, stomach, ovaries, colon, rectum, pancreas, lung (e.g., non small cell lung cancer), endometrial, non-Hodgkin's B-cell lymphoma, vagina or thyroid, sarcoma, glioblastomas, multiple myeloma or or gastric gastrointestinal cancer, especially colon carcinoma or colorectal adenoma or a tumor of the neck and head, an epidermal hyperproliferation, psoriasis, prostate hyperplasia, neuroendicrine, a neoplasia, a neoplasia of epithelial character, lymphomas, a mammary carcinoma or a leukemia.

8-12. (canceled)

13. A pharmaceutical composition comprising the pharmaceutical combination according to claim 1.

14. A pharmaceutical combination comprising 2-methyl-2-[4-(3-methyl-2-oxo-8-quinolin-3-yl-2,3-dihydro-imidazo[4,5-c]quinolin-1-yl)-phenyl]-propionitrile (Compound A) and an mTOR inhibitor selected from the group consisting of RAD rapamycin (sirolimus) and derivatives/analogs thereof such as everolimus (or RAD001); CCI-779 and Deferolimus (AP-23573/MK-8669), wherein the active ingredients are present in each case in free form or in the form of a pharmaceutically acceptable salt, and optionally at least one pharmaceutically acceptable carrier, for simultaneous, separate or sequential use for the treatment of benign or malignant tumor, carcinoma of the breast, brain, kidney, liver, adrenal gland, bladder, stomach, ovaries, colon, rectum, pancreas, lung (e.g., non small cell lung cancer), endometrial, non-Hodgkin's B-cell lymphoma, vagina or thyroid, sarcoma, glioblastomas, multiple myeloma or gastric or gastrointestinal cancer, colon carcinoma or colorectal adenoma or a tumor of the neck and head, an epidermal hyperproliferation, psoriasis, prostate hyperplasia, neuroendicrine, a neoplasia, a neoplasia of epithelial character, lymphomas, a mammary carcinoma or a leukemia, wherein Compound A is administered to a subject in need thereof in an amount between about 1 nM to about 100 nM or about 9.5×10⁻⁸ to about 9.5×10⁻⁶ Mole/kg or about 3 to about 315 mg/subject per daily dose.

15. A pharmaceutical combination according to claim 13, wherein the allosteric mTOR inhibitor compound is everolimus (RAD001) which is administered to a subject in need thereof in an amount between administered from about 0.001 nM to about 17.8 nM or from about 8.5×10⁻¹² Mole/kg to about 1.5×10⁻⁷ Mole/kg, or from about 0.00056 mg/subject to about 10 mg/subject per daily dose.

16. A method for improving efficacy of the treatment of a mammalian target of rapamycin (mTOR) kinase dependent proliferative disease comprising administering a combination comprising a compound of formula (I) according to claim 1 or a tautomer thereof, or a pharmaceutically acceptable salt, or a hydrate or solvate thereof, and at least one allosteric mTOR inhibitor compound to subject in need thereof, wherein the compound of formula (I) is administered to a subject in need thereof in an amount between about 1 nM to about 100 nM or about 9.5×10⁻⁸ to about 9.5×10⁻⁶ Mole/kg or about 3 to about 315 mg/subject per daily dose.

17. A method for treating or preventing a proliferative disease comprising administering to a subject in need thereof (a) a therapeutically effective amount of a compound of formula (I) according to claim 1 or a tautomer thereof, or a pharmaceutically acceptable salt, or a hydrate or solvate thereof, and (b) a therapeutically effective amount of at least one allosteric mTOR inhibitor compound and optionally at least one pharmaceutically acceptable carrier, wherein the compound of formula (I) is administered in an amount between about 1 nM to about 100 nM or about 9.5×10⁻⁸ to about 9.5×10⁻⁶ Mole/kg or about 3 to about 315 mg/subject per daily dose.

18. A method according to claim 17, wherein the compound of formula (I) is 2-methyl-2-[4-(3-methyl-2-oxo-8-quinolin-3-yl-2,3-dihydro-imidazo[4,5-c]quinolin-1-yl)-phenyl]-propionitrile (Compound A) or its monotosylate salt.

19. A method according to claim 17, wherein the mTOR inhibitor is selected from RAD rapamycin (sirolimus) and derivatives/analogs thereof such as everolimus (or RAD001); CCI-779 and Deferolimus (AP-23573/MK-8669).

20. A method according to claim 17, wherein the allosteric mTOR inhibitor compound is everolimus (RAD001) which is administered to a subject in need thereof in an amount between administered from about 0.001 nM to about 17.8 nM or from about 8.5×10⁻¹² Mole/kg to about 1.5×10⁻⁷ Mole/kg, or from about 0.00056 mg/subject to about 10 mg/subject per daily dose.

21. A method according to claim 17, wherein the proliferative disease is a benign or malignant tumor, carcinoma of the breast, brain, kidney, liver, adrenal gland, bladder, stomach, ovaries, colon, rectum, pancreas, lung (e.g., non small cell lung cancer), endometrial, non-Hodgkin's B-cell lymphoma, vagina or thyroid, sarcoma, glioblastomas, multiple myeloma or gastric or gastrointestinal cancer, especially colon carcinoma or colorectal adenoma or a tumor of the neck and head, an epidermal hyperproliferation, psoriasis, prostate hyperplasia, neuroendocrine, a neoplasia, a neoplasia of epithelial character, lymphomas, a mammary carcinoma or a leukemia.
